(19) United States
(12) Patent Application Publication
(10) Pub. No.: US 2024/0290418 A1
(43) Pub. Date: Aug. 29, 2024

(54) TCR-REPERTOIRE FRAMEWORK FOR MULTIPLE DISEASE DIAGNOSIS

(71) Applicant: THE BOARD OF REGENTS OF THE UNIVERSITY OF TEXAS SYSTEM, Austin, TX (US)

(72) Inventor: Bo LI, Austin, TX (US)

(73) Assignee: THE BOARD OF REGENTS OF THE UNIVERSITY OF TEXAS SYSTEM, Austin, TX (US)

(21) Appl. No.: 18/571,515

(22) PCT Filed: Jun. 17, 2022
(86) PCT No.: PCT/US2022/034068

§ 371 Date: Dec. 18, 2023

Related U.S. Application Data
(60) Provisional application No. 63/202,716, filed on Jun. 22, 2021.

(51) Int. Cl. G16B 15/00; G16B 40/20; G16H 50/20

(52) U.S. Cl. CPC G16B 15/00; G16B 40/20; G16H 50/20

(57) ABSTRACT

A novel method of geometric isometry based antigen-specific TCR alignment (GIANA) is described herein. GIANA is an antigen-specific TCR clustering method that is able to efficiently handle tens of millions of sequences. GIANA achieved higher sensitivity and precision than all existing methods, and is able to retrieve TCRs specific to known antigens with high accuracy. The ultra-large-scale TCR clustering and fast query of novel samples also enabled a novel reference-based repertoire classification framework. GIANA can also analyze single cell RNA-seq data with TCR regions solved, and it is possible to query TCRs from unknown data against the large database of TCR repertoire samples in the public domain, and provide new insights over shared antigen-specificity. GIANA is applicable to cluster or query large B cell receptor sequencing data as well.

### CROSS-REFERENCE TO RELATED APPLICATIONS

This application claims benefit of priority under 35 U.S.C. § 119(e) of U.S. Provisional Application No. 63/202,716, filed Jun. 22, 2021. The disclosure of the prior application is considered part of and is herein incorporated by reference in the disclosure of this application in their entirety.

### TECHNICAL FIELD

The present disclosure generally relates to immune-repertoire based disease diagnosis technology, and more particularly to a novel system and method for efficiently grouping similar T cell receptor (TCR) sequences and diagnosing a patient with a disease and determining his/her disease status with a peripheral blood TCR repertoire.

### BACKGROUND

Adaptive immune repertoire is an important regulator of diverse human diseases, and over 10,000 TCR repertoire sequencing (TCR-seq) samples have been generated in the recent years. However, interpretation of TCR data has been hindered by the scarcity of known antigen-specificities. Recent studies demonstrated that similarity in the TCR hypervariable complementarity-determining region 3 (CDR3) implicates structural resemblance for antigen recognition. Therefore, clustering of similar CDR3s has become an important way to identify antigen-specific receptors.

### SUMMARY

Methods, systems, and apparati for improving computational efficiency for T-cell receptor (TCR) comparisons are described herein. In an example, complementary determining region 3 (CDR3) sequences may be identified from a reference TCR sequence (TCR-seq) dataset. The reference TCR-seq dataset may consist of TCRs specific to only one epitope. Each of the CDR3 sequences from the reference TCR-seq dataset may be encoded into numeric vectors, the numeric vectors corresponding to a sequence of amino acids in each of the CDR3 sequences. The numeric vectors to may be converted to coordinates in a high-dimensional Euclidean space. A predictive model may be generated using a neural network. The neural network may learn to generate a tree data structure of the numeric vectors based on relative distances of the coordinates and may grouping the coordinates into pre-clusters based on the relative distances. The CDR3 sequences in the pre-clusters may be filtered using one or more criteria to reduce noise. Antigen-specific CDR3 clusters may be identified from the filtered pre-clusters.

In another example, unknown TCR-seq samples may be queried against existing reference data to diagnose a patient with a disease and determining his/her disease status with a peripheral blood TCR repertoire. The identifying, encoding, converting, generating, and filtering steps described above may also be performed on a query TCR-seq dataset. The query TCR-seq dataset may have no known antigen-specific TCR information. The filtered pre-clusters from the query TCR-seq dataset may be compared to the antigen-specific CDR3 cluster. The filtered pre-clusters from the query TCR-seq dataset may be determined to match the antigen-specific CDR3 clusters.

In another example, a large TCR database may be queried and grouped into TCR clusters of common antigen specificity. A nearest neighbor search may be performed using one or more TCR dissimilarity metrics to find pairs of TCRs with common antigen specificity. The one or more TCR dissimilarity metrics may include one or more of a Smith-Waterman distance and an embedding in a high-dimensional Euclidean space; or any other distance or dissimilarity metric.

### BRIEF DESCRIPTION OF THE DRAWINGS

The drawings described below are for illustration purposes only. The drawings are not intended to limit the scope of the present disclosure.

FIG. 1 is a diagram of a system, according to some embodiments of the present disclosure;

FIG. 2 is a block diagram illustrating components for performing the methods described herein, according to some embodiments of the present disclosure;

FIG. 3 is a flowchart illustrating the GIANA analysis of reference TCR-seq data, according to some embodiments of the present disclosure;

FIG. 4 is a chart illustrating the performance of multidimensional scaling (MDS) based isometric embedding, according to some embodiments of the present disclosure;

FIGS. 5A-5F show charts illustrating a comparison of G6-encoded isometric distances for CDR3 strings with Smith-Waterman alignment scores, according to some embodiments of the present disclosure;

FIG. 6 is a graphic illustrating an overview of the geometric isometry based antigen-specific TCR alignment (GIANA) workflow, according to some embodiments of the present disclosure;

FIG. 7 is a graphic illustrating an overview of the GIANA workflow using stacked MDS vectors (GIANAsv), according to some embodiments of the present disclosure;

FIG. 8 is a chart showing a comparison of time complexity for the different TCR clustering algorithms, according to some embodiments of the present disclosure;

FIG. 9 is a chart showing memory usage of the different TCR clustering algorithms when evaluating time complexity, according to some embodiments of the present disclosure;

FIG. 10 is a chart illustrating clustering precision, according to some embodiments of the present disclosure;

FIG. 11 is a chart illustrating clustering sensitivity, according to some embodiments of the present disclosure;

FIG. 12 is chart illustrating a normalized mutual information (NMI) comparison between four methods of TCR clustering;

FIG. 13 is a chart illustrating precision-recall curves measuring the performance of GIANA in a range of parameter settings;

FIG. 14 is a chart illustrating precision-recall curves measuring the performance of GIANA using different substitution matrixes;

FIGS. 15A-15F are charts illustrating the sensitivity and specificity of GIANA when applied to large and noisy TCR sequence (TCR-seq) samples, according to some embodiments of the present disclosure;

FIGS. 16A-16B shows sensitivity and specificity estimations for GLIPH2, according to some embodiments of the present disclosure;

FIG. 16C shows positive prediction value (PPV) estimations for GLIPH2 and GIANA, according to some embodiments of the present disclosure;

FIG. 17 is a diagram illustrating fast GIANA query based on isometric transformation, according to some embodiments of the present disclosure;

FIG. 18 is a chart illustrating a time complexity evaluation of GIANA query module using reference/query data with different number of TCRs, according to some embodiments of the present disclosure;

FIG. 19 is a chart illustrating a degree of separation of query COVID-19 patients from healthy controls by clustering against reference datasets, according to some embodiments of the present disclosure;

FIGS. 20A-20C are charts illustrating receiver operating characteristic (ROC) curves using COVID-19 fraction as the single predictor, according to some embodiments of the present disclosure;

FIGS. 21A-21D are charts illustrating a coefficient of variance of COVID-19 fractions with different number of reference TCRs, according to some embodiments of the present disclosure;

FIGS. 22A-22D are graphic representations of similarities of the TCR-seq samples based on TCR co-clustering, according to some embodiments of the present disclosure;

FIGS. 23A-23B are beeswarm plots illustrating the distributions of TCR clonal frequencies of different categories, according to some embodiments of the present disclosure;

FIGS. 24A-24B are graphs illustrating dynamic changes of TCR clonal frequencies during the course of Severe Acute Respiratory Syndrome Coronavirus-2 (SARS-CoV-2) infection, according to some embodiments of the present disclosure;

FIGS. 25A-25F are charts illustrating ROC curves using a leave-one-out validation approach for disease fractions calculated from co-clustered TCRs, according to some embodiments of the present disclosure;

FIGS. 26A-26F are charts illustrating ROC curves using a more stringent method for disease fractions calculated from co-clustered TCRs, according to some embodiments of the present disclosure;

FIG. 27 is a chart illustrating cross-cohort similarity of reference TCR-seq samples, according to some embodiments of the present disclosure;

FIGS. 28A-28D are violin plots illustrating the distribution of class fractions of cancer, COVID-19, multiple-sclerosis (MS) patients, and healthy controls (HCs), according to some embodiments of the present disclosure; and

FIGS. 29A-29F are charts illustrating ROC curves using disease class fractions as single predictor for pairwise separation 4 disease classes, according to some embodiments of the present disclosure.

### DETAILED DESCRIPTION

A number of conventional studies have applied TCR clustering to investigate antigen-specific T cell responses during disease progression or immunotherapy treatments. It is speculated that integrating a large number of TCR-seq samples from multiple studies will result in more insights into immune-disease interactions and create novel opportunities for prognosis and diagnosis. Nonetheless, high clustering specificity requires pairwise Smith-Waterman alignment on both the CDR3 sequences and the TCR variable gene (TRBV) alleles, which has quadratic computational complexity that usually cannot scale up to the scale of TCR repertoire samples (≥100K sequences). Motif-based clustering achieves higher speed, but has much lower specificity. Therefore, none of the existing TCR clustering methods are suitable to analyze large cohorts of TCR-seq samples.

Unsupervised TCR clustering is a fundamental analysis of immune repertoire data. In the ideal scenario, all TCRs specific to the same epitope should be included in the same cluster. However, this is not feasible for sequence similarity or motif based clustering approach, due to the putative diversity in TCR sequences of shared specificity. Such diversity is caused by the distinct docking strategies of T cell receptors. For example, TCRs specific to the influenza GIL epitope usually contain the classic RSS/RSA motif in the CDR3 region, yet a related study reported that the LGGW motif also elicits strong binding to GIL from a different direction. Such structural variation cannot be captured by simple Smith-Waterman alignment, or motif grouping. Consequently, CDR3s with dissimilar motifs will be fragmented into smaller clusters despite their shared specificity, which is a common limitation to the current methods.

To address this challenge, a novel framework was developed to transform the CDR3 sequences and convert the sequence alignment and clustering problem into a nearest neighbor search in high-dimensional Euclidean space. This transformation may significantly improve the computational efficiency for TCR pairwise comparisons, and may scale up to 106 to 107 sequences. By pooling thousands of TCR repertoire samples, which conventional systems and methods are unable to do, the novel method described herein may be able to identify novel disease-associated TCRs. As described more herein, this may open new avenues for a new multi-disease diagnosis platform.

The present disclosure will now be described more fully hereinafter with reference to the accompanying drawings, which form a part hereof, and which show, by way of non-limiting illustration, certain examples. Subject matter may, however, be described in a variety of different forms and, therefore, covered or claimed subject matter is intended to be construed as not being limited to any examples set forth herein. Among other things, subject matter may be described as methods, devices, components, or systems. Accordingly, examples may take the form of hardware, software, firmware or any combination thereof (other than software per se). The following detailed description is, therefore, not intended to be taken in a limiting sense.

In general, terminology may be understood at least in part from usage in context. For example, terms, such as “and”, “or”, or “and/or,” as used herein may include a variety of

meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The present disclosure is described below with reference to block diagrams and operational illustrations of methods and devices. It is understood that each block of the block diagrams or operational illustrations, and combinations of blocks in the block diagrams or operational illustrations, may be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession may be executed substantially concurrently or the blocks may sometimes be executed in the reverse order, depending upon the functionality/acts involved.

For the purposes of this disclosure a non-transitory computer readable medium (or computer-readable storage medium/media) stores computer data, which data can include computer program code (or computer-executable instructions) that is executable by a computer, in machine readable form. By way of example, and not limitation, a computer readable medium may comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, CD-ROM, DVD, or other optical storage, cloud storage, magnetic cassettes, magnetic tape, magnetic disk storage or other magnetic storage devices, or any other physical or material medium which can be used to tangibly store the desired information or data or instructions and which can be accessed by a computer or processor.

For the purposes of this disclosure the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

For the purposes of this disclosure, a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router (WR) mesh, or 2nd, 3rd, 4th or 5th

generation (2G, 3G, 4G or 5G) cellular technology, Bluetooth, 802.11b/g/n, or the like. Network access technologies may enable wide area coverage for devices, such as client devices with varying degrees of mobility, for example.

In short, a wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

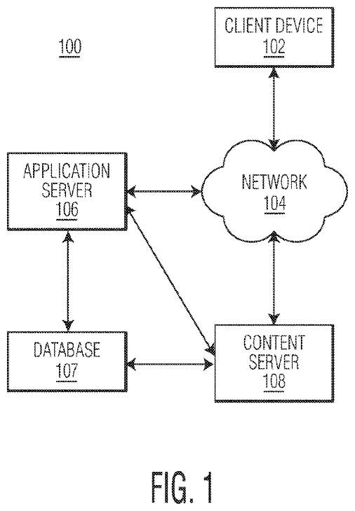

Referring now to FIG. 1, a system 100 is shown. FIG. 1 illustrates components of a general environment in which the systems and methods discussed herein may be practiced. Not all the components may be required to practice the disclosure, and variations in the arrangement and type of the components may be made without departing from the spirit or scope of the disclosure.

The system 100 of FIG. 1 includes network 104, which as discussed above, may include, but is not limited to, a wireless network, a local area network (LAN), wide area network (WAN), the Internet, or a combination thereof.

The network 104 may be connected, for example, to one or more client devices 102, an application server 106, a content server 108, and a database 107 and their components with another network or device. The network 104 may be configured as a variety of wireless sub-networks that may further overlay stand-alone ad-hoc networks, and the like, to provide an infrastructure-oriented connection for the one or more client devices 102, the application server 106, the content server 108, and the database 107. The network 104 may be configured to employ any form of computer readable media or network for communicating information from one electronic device to another.

The one or more client devices 102 may, for example, include a desktop computer or a portable device, such as a cellular telephone, a smart phone, a display pager, a radio frequency (RF) device, an infrared (IR) device, a Near Field Communication (NFC) device, a Personal Digital Assistant (PDA), a handheld computer, a tablet computer, a phablet, a laptop computer, a set top box, a wearable computer, smart watch, an integrated or distributed device combining various features, such as features of the forgoing devices, or the like.

The one or more client devices 102 may also include at least one client application that is configured to receive content from another computing device. The one or more client devices 102 may communicate over the network 104 with other devices or servers, and such communications may include sending and/or receiving messages, generating and providing TCR data, searching for, viewing and/or sharing TCR data, or any of a variety of other forms of communications. The one or more client devices 102 may be capable of processing or storing signals, such as in memory as physical memory states, and may, therefore, operate as a server

The application server 106 and the content server 108 may include one or more devices that are configured to provide and/or generate any type or form of content via a network to another device. Devices that may operate as the application server 106 and/or the content server 108 may include personal computers, desktop computers, multiprocessor systems, microprocessor-based or programmable consumer electronics, network PCs, servers, and the like. The application server 106 and the content server 108 may store various types of data related to the content and services provided by each device in the database 107.

Users (e.g., patients, doctors, technicians, and the like) may be able to access services provided by the application server 106 and the content server 108. This may include, for example, application servers, authentication servers, search servers, exchange servers, via the network 104 using the one or more client devices 102. Thus, the application server 106, for example, may store various types of applications and application related information including application data and user profile information.

Although FIG. 1 illustrates the application server 106 and the content server 108 as single computing devices, respectively, the disclosure is not so limited. For example, one or more functions of the application server 106 and the content server 108 may be distributed across one or more distinct computing devices. In another example, the application server 106 and the content server 108 may be integrated into a single computing device without departing from the scope of the present disclosure.

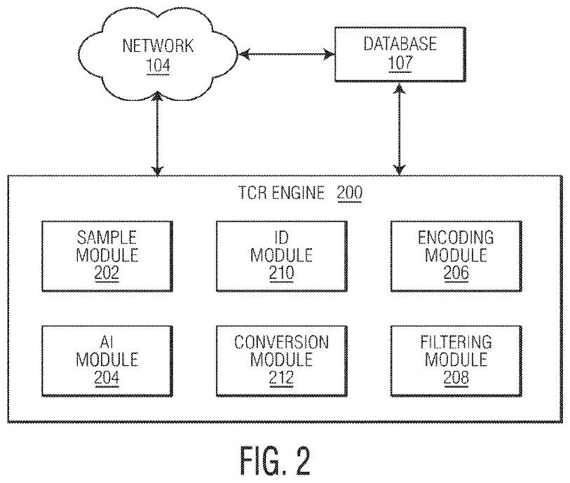

Referring now to FIG. 2, a block diagram illustrating components for performing the methods described herein is shown. FIG. 2 includes a TCR engine 200, the network 104, and the database 107. The TCR engine 200 may be a special purpose machine or processor and may be hosted by one or more of the application server 106, the content server 108, a web server, a third party server, a user's computing device, and the like.

In an example, the TCR engine 200 may be a conventional personal computer, and the methods described below may be performed using a single thread on a CPU. In another example, when clustering reference data of 10 million sequences, the TCR engine 200 may be a high-performance computing (HPC) super cluster (e.g., with 128G memory allocation and 8 CPU nodes).

The TCR engine 200 may be a stand-alone application that executes on a device (e.g., a user device or system/web-connected server/device). In another example, the TCR engine 200 may function as an application installed on the device and/or a web-based application accessed by the device over a network. The TCR engine 200 may be installed as an augmenting script, program or application (e.g., a plug-in or extension) to another application, such as, for example, a health care application that aggregates and shares patient related data.

The database 107 may be any type of database or memory, and may be associated with a server on a network (e.g., the application server 106 and the content server 108) or a user's device (e.g., the one or more client devices 102). The database 107 may include a dataset of data and metadata associated with local and/or network information related to users, services, applications, content and the like. Such information may be stored and indexed in the database 107 independently and/or as a linked or associated dataset. As discussed herein, it should be understood that the data (and metadata) in the database 107 can be any type of information and type, whether known or to be known, without departing from the scope of the present disclosure.

The database 107 may store data for users (e.g., user data. The stored user data may include, for example, information associated with reference TCR-seq data, a patient's cancer diagnosis, patient's chromosomal information, patient's DNA information, patient's blood information, patient demographic information, patient biographic information, and the like, or some combination thereof.

The data (and metadata) in the database 107 may be any type of information related to TCR-seq data, a patient, doctor, content, a device, an application, a service provider, a content provider, whether known or to be known, without departing from the scope of the present disclosure.

The data stored in the database 107 may be encrypted, for example, using a 256-bit encryption, such that the data is private and controlled according to Health Insurance Portability and Accountability Act of 1996 (HIPPA).

The database 107 may store and index the information as linked set of data and metadata, where the data and metadata relationship can be stored as the n-dimensional vector. Such storage can be realized through any known or to be known vector or array storage, including, but not limited to, a hash tree, queue, stack, VList, or any other type of known or to be known dynamic memory allocation technique or technology. It should be understood that any known or to be known computational analysis technique or algorithm, such as, but not limited to, cluster analysis, data mining, Bayesian network analysis, Hidden Markov models, artificial neural network analysis, logical model and/or tree analysis, and the like, and be applied to determine, derive or otherwise identify vector information for patients and/or health care providers.

As discussed above with reference to FIG. 1, the network 104 may be any type of network such as, but not limited to, a wireless network, a local area network (LAN), wide area network (WAN), the Internet, or a combination thereof. The network 104 may facilitate connectivity of the TCR engine 200 and the database 107 of stored resources. Indeed, as illustrated in FIG. 2, the TCR engine 200 and the database 107 may be directly connected by any known or to be known method of connecting and/or enabling communication between such devices and resources.

The principal processor, server, or combination of devices that include hardware programmed in accordance with the special purpose functions herein may be referred to for convenience as TCR engine 200. The TCR engine 200 may include a sample module 202, an AI module 204, an encoding module 206, a filtering module 208, an identification (ID) module 210, and a conversion module 212. The engine(s) and modules discussed herein are non-exhaustive, as additional or fewer engines and/or modules (or sub-modules) may be applicable to the examples of the systems and methods discussed. The operations, configurations and functionalities of each module, and their role within examples of the present disclosure are discussed below.

The principles described herein may be embodied in many different forms. T cells reactive to antigens are central mediators of immunity against various diseases and key targets of immunotherapies, yet as most of cancer antigens are unknown, experimental detection of cancer-associated T cells remains difficult. The recent development of deep immune repertoire sequencing (TCR-seq) technology has placed an additional emphasis on the identification of such T cells, as it may open new opportunities for non-invasive clinical diagnosis, prognosis and longitudinal immune monitoring of cancer patients. However, human immune repertoire contains public T cells, naïve T cells, and memory/effector T cells specific to diverse antigens, and this complexity adds to the challenges conventional systems are unable to solve (e.g., to identify cancer-associated T cells in the TCR-seq data).

Previous studies on the TCR repertoires of cancer patients reported that simple statistics, such as diversity and clonality, are associated with clinical outcome under certain conditions, substantiating the utilities of repertoire data as a potential prognostic factor. However, with the fast advancement of immunotherapies and rapid accumulation of TCR-seq data, more computational tools are required to bridge the gap between basic immunogenomics research and clinical applications beneficial to cancer patients.

The disclosed systems and methods provide these needed tools through a novel framework executing ensemble machine learning software (referred to as TCRboost) that provides for de novo prediction of cancer-associated immune repertoires using the (3 chain TCR-seq data.

Grouping of similar TCR sequences implicates shared antigen-specificity, and can be used to discover novel therapeutical targets. Conventional methods suffer from high computational expenses that cannot scale up to the magnitude of immune repertoire datasets. Geometric isometry based antigen-specific TCR alignment (GIANA), described herein, may be used to close the gap between speed and prediction accuracy, with better precision and sensitivity than conventional methods (e.g., TCRdist) at approximately 600 times of its speed. GIANA may also allow ultrafast query of large reference cohorts, processing over 100 billion sequence comparisons within 3 minutes. In an example, GIANA may be able to compare 104 TCRs against 107 reference sequences within 3 minutes. Applying GIANA to cluster large-scale TCR datasets may reveal novel insights of disease-specific receptors and provide a new solution to the repertoire classification task. Query of unseen TCR-seq samples against existing references using GIANA may achieve high accuracies and may be used to differentiate cancer, infectious disease, and autoimmune disorders. GIANA may be used as a TCR-based non-invasive multi-disease diagnostic platform.

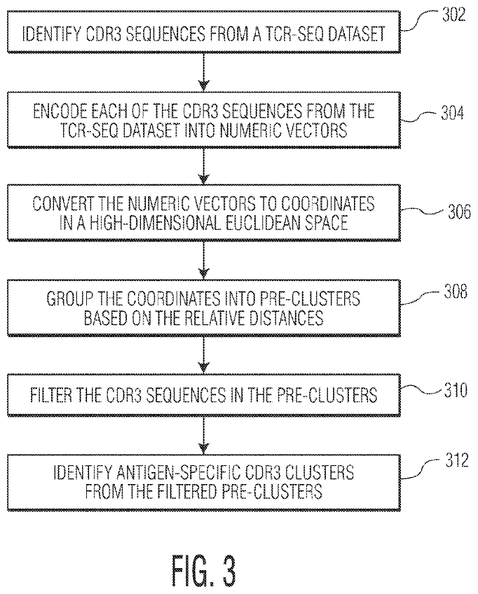

Referring now to FIG. 3, a flowchart illustrating the GIANA analysis of reference TCR-seq data is shown. It should be noted that the steps shown in FIG. 3 may be performed by the TCR Engine 200, described above with reference to FIG. 2.

In step 302, the sample module 202 may identify CDR3 sequences from a TCR-seq dataset. The sample module 202 may receive the TCR-seq dataset from, for example, the database 107. In an example, the TCR-seq dataset may include reference TCR-seq dataset consisting of TCRs specific to only one epitope. In step 304, the encoding module 206 may encode each of the CDR3 sequences from the TCR-seq dataset into numeric vectors. The numeric vectors may correspond to a sequence of amino acids in each of the CDR3 sequences.

In step 306, the conversion module 212 may convert the numeric vectors to coordinates in a high-dimensional Euclidean space. In step 308, the AI module 204 may generate a predictive model using a neural network. The neural network may learn to generate a tree data structure of the numeric vectors based on relative distances of the coordinates and may then group the coordinates into pre-clusters based on the relative distances. In step 310, the filtering module 208 may filter the CDR3 sequences in the pre-clusters. In step 312, the ID module 210 may identify antigen-specific CDR3 clusters from the filtered pre-clusters. The GIANA process is described in additional detail below.

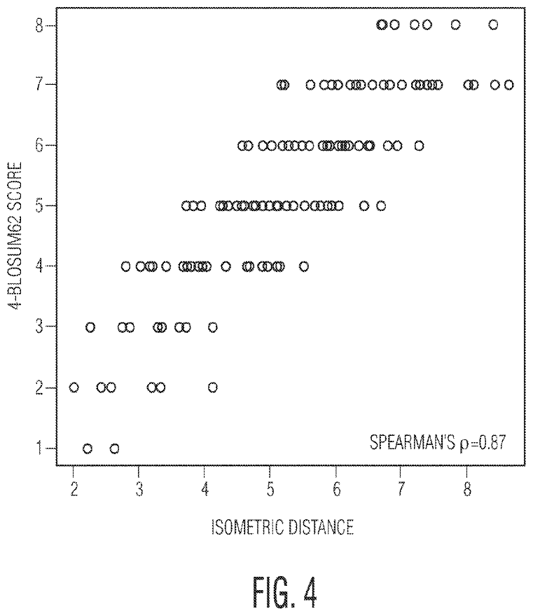

Referring now to FIG. 4, a chart illustrating the performance of multidimensional scaling (MDS) based isometric embedding is shown. GIANA begins with an approximated solution to the isometric embedding of BLOSUM62 matrix using MDS, which may generate a vector for each of the 20 amino acids that make up proteins. Each amino acid may be represented by a numeric vector. In an example, all 20 vectors may be calculated using a non-metric multi-dimensional scaling algorithm available in Python. The Euclidean distances between each pair of amino acids may be calculated, resulting in a total of 190 pairs. All 190 distances (squared) may be visually compared to a corresponding score in the BLOSUM62 matrix. The distances squared may be compared to the corresponding transformed BLOSUM62 dissimilarity scores (4-BLOSUM62 scores, with diagonal set 0). For example, amino acids W and F may have a BLOSUM62 score of 1. Their distance by calculating the isometric embedding vectors may be approximately 2.3. Hence, a point (2.3, 1) me be displayed on the scatter plot shown in FIG. 4.

Spearman's correlation may be calculated to evaluate the similarity of the two measures. Subsequently, CDR3 strings may be modeled as serial non-commuting linear transformations on the MDS vectors, and may be represented as coordinates in the high-dimensional space. The unitary transformation matrix may be an element of the cyclic group of any order that is large enough relevant to the typical length of a CDR3 sequence, such as a 6-order cyclic group (G₆), which may produce near-perfect linear correlations between the Euclidean distances of a pair of strings and their alignment scores.

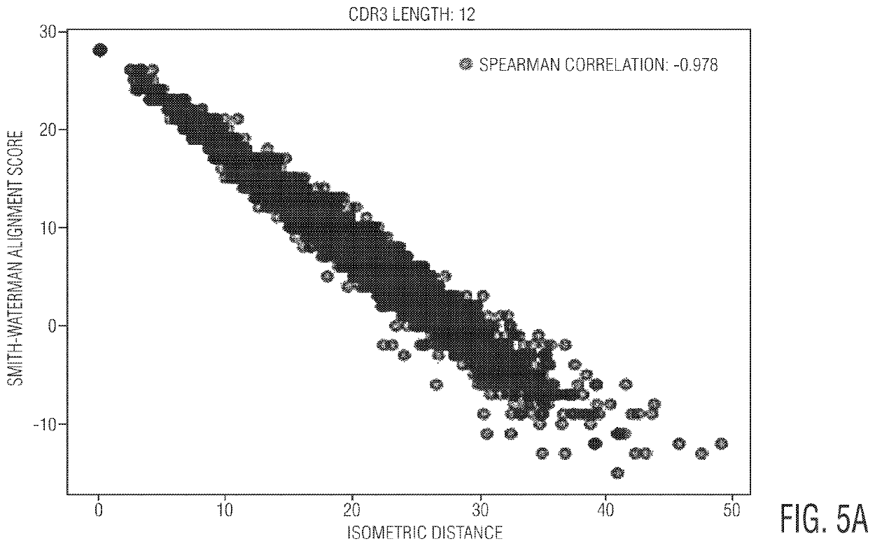

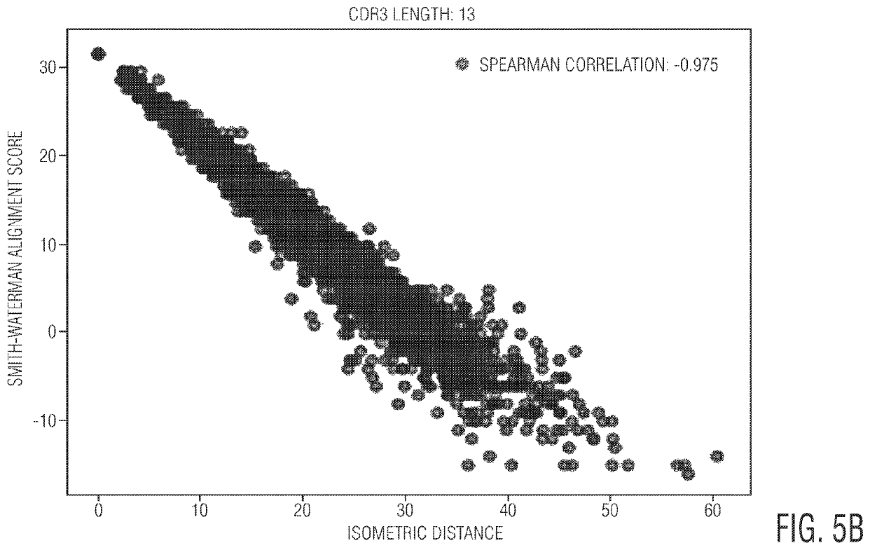

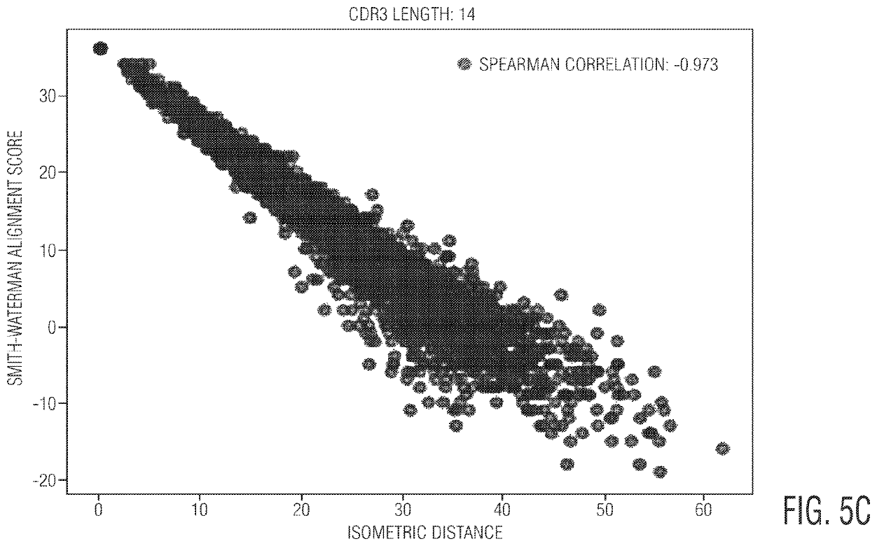

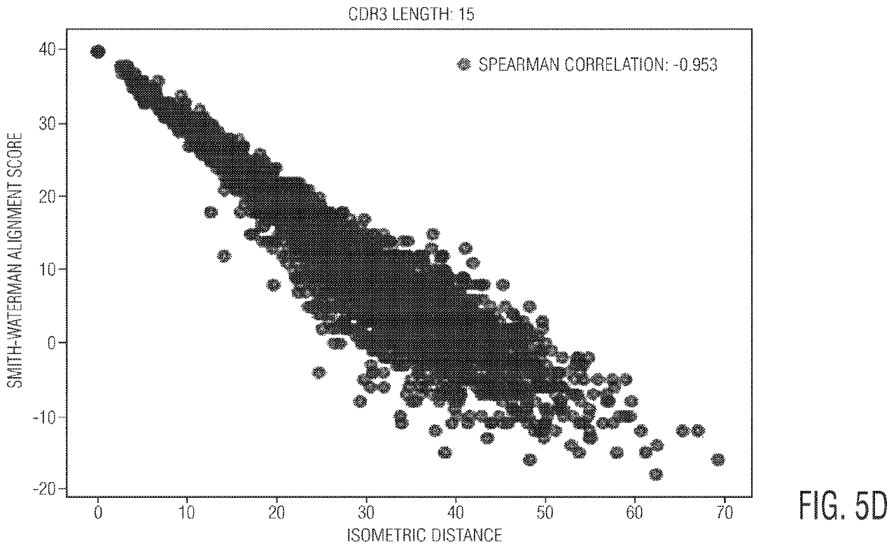

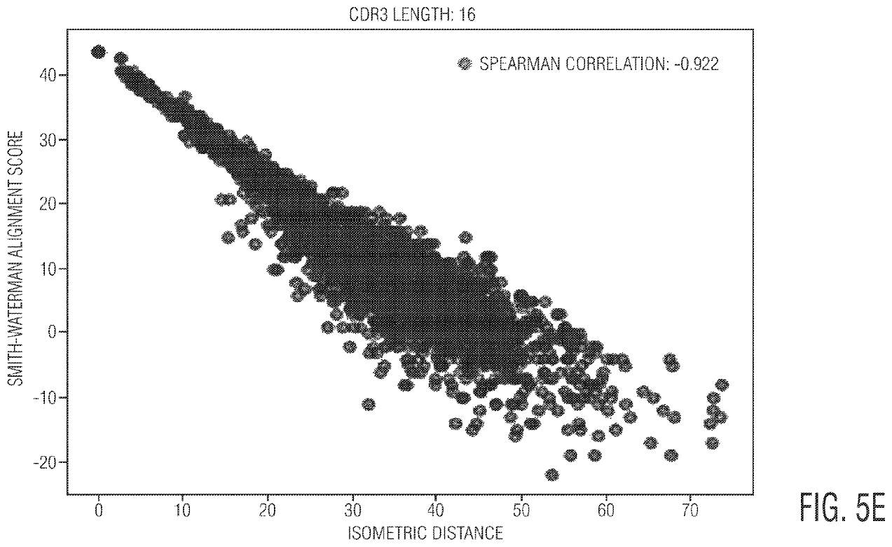

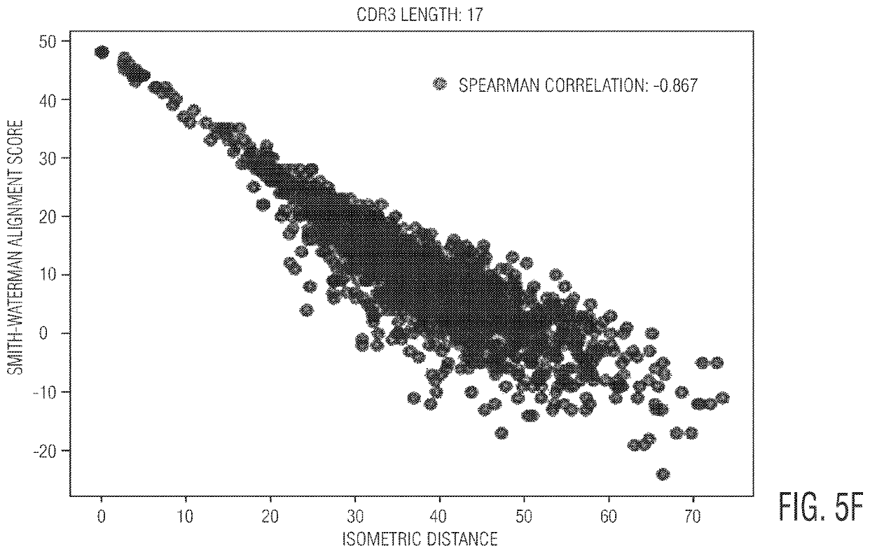

Referring now to FIGS. 5A-5F, charts illustrating a comparison of G6-encoded isometric distances for CDR3 strings with Smith-Waterman alignment scores are shown. At a default isometric distance cutoff (−t) of 10, all TCR pairs with high Smith-Waterman alignment scores are included in downstream clusters. FIGS. 5A-5F show an analysis for CDR3s with a length of 12 to 17 amino acids, respectively. In each chart, the x-axis axis may represent isometric distance (e.g., Euclidean distances squared) between pairs of CDR3s and the y-axis may represent corresponding Smith-Waterman alignment scores using BLOSUM62 as a substitution matrix. The isometric distance may be defined as the squared Euclidean distance between a pair of numeric vectors after G6 encoding. FIGS. 5A-5F show an analysis of 10,000 CDR3 sequences which have been split into different length categories (i.e., 12 to 17 amino acids). The numeric vector representations for all the sequences in each length category may be obtained and the pairwise distances may be calculated. The sequence similarity of each pair of CDR3 sequences may be assessed using the classic Smith-Waterman alignment algorithm, which may rely on an amino acid substitution matrix (e.g., BLOSUM62). Higher alignment scores indicate higher sequence similarity. For two identical sequences, the maximum score of 4*length is reached. The Spearman's correlation values are shown as negative because higher alignment scores implicate higher similarity, which correspond to smaller distances. This is different from the dissimilarity scores used in FIG. 4.

Fast, index-based nearest neighbor search and recursive centroid grouping may be performed on the coordinates to identify CDR3 pre-clusters (i.e., TCRs of high similarity and putatively common antigen specificity) with high computational efficiency. Methods for the nearest neighbor search may include one or more conventional methods such as: Facebook AI Similarity Search (FAISS), Navigable Small World (NSW), Hierarchal Navigable Small World (HNSW), PyNNDescent, and Annoy. TCR dissimilarity measures used for the nearest neighbor search may include one or more of: Smith-Waterman distance, an embedding in a high-dimensional Euclidean space, or any other distance or dissimilarity metric used to estimate the common antigen specificity of two TCRs. The CDR3 pre-clusters may be subsequently filtered for matched TRBV alleles and high Smith-Waterman alignment scores using a k-mer guided search table, to produce final TCR clusters as output.

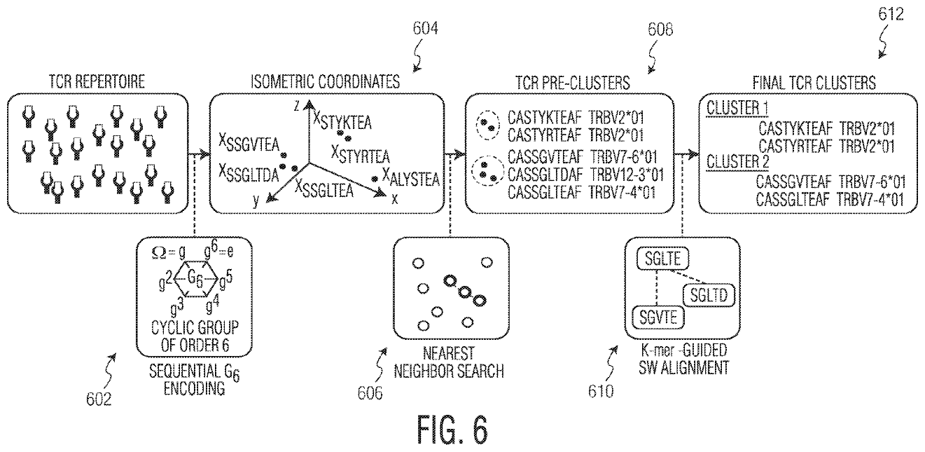

Referring now to FIG. 6, a graphical illustration of the GIANA workflow is shown. In step 602, the GIANA process may begin with encoding of short CDR3 peptide sequences into numeric vectors through a sequence of unitary transformations. As described below in additional detail, the transformation may involve an element of 6^{th} order cyclic group. In step 604, each encoded CDR3 sequence may be projected to high-dimensional Euclidean space. In step 606, a fast nearest neighbor search may be performed. In step 608, iterative centroid clustering may be performed. In step 610, filtering steps may be performed to match the TRBV gene alleles and remove pairs with low alignment scores. In step 612, final TCR clusters may be output.

Additionally or alternatively to the G₆ transformation performed in step 602, a similar method using stacked MDS vectors (GIANAsv) as coordinates of the input CDR3 strings may be used.

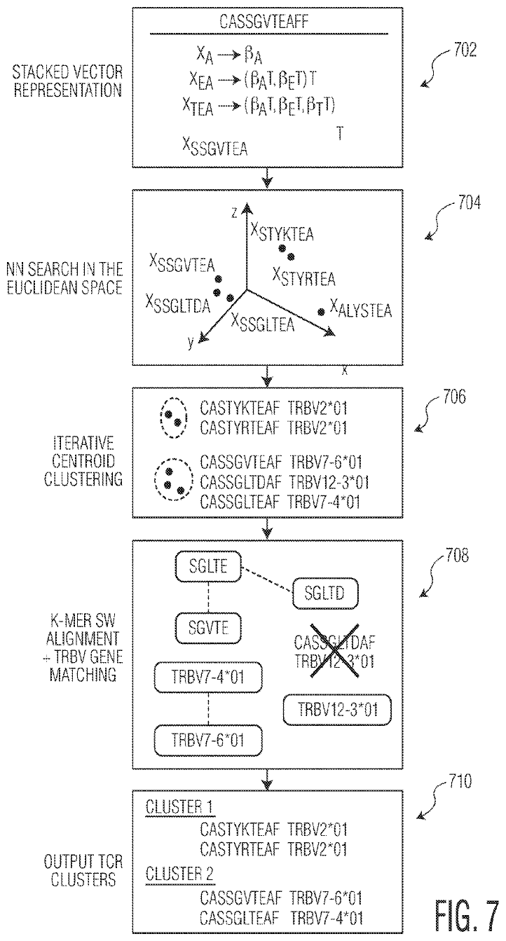

Referring now to FIG. 7, a graphical illustration of the GIANAsv workflow is shown. In step 702, input sequences may be encoded as concatenated vectors of all the amino acids in the string. Apart from the encoding of input sequence, the other processing steps may be similar to GIANA. For example, in step 704, each encoded CDR3 sequence may be projected to high-dimensional Euclidean space, and a fast nearest neighbor search may be performed. In step 706, iterative centroid clustering may be performed. In step 708, filtering steps may be performed to match the TRBV gene alleles and remove pairs with low alignment scores. In step 710, final TCR clusters may be output. The GIANA and GIANAsv processes are described in further detail herein.

In an example, the GIANA process may be used to identify and classify antigen-specific CDR3 sequences from reference TCR-seq data. TCR repertoire sequencing samples may be accessed from one or more databases, such as the immuneACCESS database provided by Adaptive Biotechnology, which is currently the largest database of TCR-seq samples, all profiled using the immunoSEQ platform. Antigen-specific TCR and the matched antigens may be pooled from the VDJdb, the Immune Epitope Database and Analysis Resource (IEDB), and previous literature. TCRs specific to more than one epitope may be removed from the reference TCR-seq data to avoid conflicts.

A mathematical framework for isometric embedding of CDR3 sequences may be used to find a numeric representation (also the coordinates in high-dimensional space) x of any short peptide sequence s, such that, for sᵢ and sⱼ, the Euclidean distance between the two coordinates xᵢ and xⱼ:∥xᵢ−xⱼ∥, is perfectly correlated to the sequence similarity score measured by putative evolutionary substitution matrix.

This problem may be referred to as the “isometric embedding of short sequences.” This concept is introduced to solve the numeric encoding problem of the CDR3 sequences, typically with lengths ranging from 12 to 17 amino acids. A mathematical transformation of a given CDR3 sequence may be found to approximately satisfy isometry. First, an approximately isometric embedding for the BLOSUM62 matrix may be found, as described below.

Let amino acid Aᵢ be represented by βᵢ, a numeric vector in real space [symbol] ^{r}. The dimension of the real space r is determined by the rank of the Euclidean Distance Matrix (EDM). In this scenario, let M denote the dissimilarity matrix derived by BLOSUM62: M=4−BLOSUM62, and the diagonal values of M are set 0. Isometry indicates that:

$$‖ {β}_{i} - {β}_{j} ‖ = {M}_{i j} \qquad ( Equation 1 )$$

The solution to this problem exists if, and only if the EDM is flat, and the embedding space has dimension no greater than n, where n is the dimension of EDM. Unfortunately, BLOSUM62 matrix is not an EDM, since it does not satisfy the triangular rule:

$${M}_{i k} + {M}_{k j} \geq {M}_{i j} , for ∀ i , j , k \qquad ( Equation 2 )$$

Therefore, there may not be an exact isometric embedding of BLOSUM62. However, MDS may provide an approximate solution, which applies to the examples where M is not an EDM. MDS may be used to derive the embedding vectors βᵢ. With classic MDS, the maximum dimension for the embedding space is 13. A non-metric MDS calculation may be applied using the sklearn package in Python to explore dimensionality higher than 13. To maximize the embedding isometry, 2,300 training TCRs of length 14 may be selected from a TCGA dataset and pairwise SW alignment scores may be calculated. The MDS may be applied to obtain isometric embedding vectors of different dimensions, ranging from 13 to 19. For each length, the Euclidean coordinates of the CDR3 sequences may be calculated as described in the GIANA method. The pairwise distance may be compared with SW scores. The maximum score was observed with dimension 16, which may be the optimal dimension for isometric representation. This representation may achieve approximately 87% similarity to the BLOSUM matrix:

$$‖ {β}_{i} - {β}_{j} ‖ \approx {M}_{i j} \qquad ( Equation 3 )$$

Next, a numeric encoding scheme may be introduced, such that each amino acid in a CDR3 sequence may be considered as an “operator,” parable to the concept in quantum physics. In general, operator A may be a mathematical transformation to an existing wave function ϕ. The operation may be applied to wave functions denoted by the Dirac bracket: A|ϕ). One example is the angular moment operators Lₓ, L_{y}, L_{z}. The operator for amino acid i may be defined as Aᵢ, which may apply to a numeric vector x in the following way:

$${A}_{i} x := Ω \times ( x + {β}_{i} ) \qquad ( Equation 4 )$$

Here Ω is a matrix that needs to be determined. This definition emphasizes the ordering of letters in a sequence as operators are non-commuting: AᵢAⱼ≠AⱼAᵢ if i≠j. A CDR3 sequence may then be seen as one or more serial linear operations on some initial vector, β₀. To simplify calculations, let β₀=0. Therefore, after operation on the rightmost amino acid, the coordinates become:

$${A}_{i} {β}_{0} := Ω \times ( {β}_{i} + 0 ) = Ω \times {β}_{i} \qquad ( Equation 5 )$$

Several examples to illustrate the desirable qualities of Ω are described below.

In a first example, two amino acids sequences may be off by one amino acid (e.g., a single mismatch). For example, sequence s₁=AₖAᵢ, and sequence s₂=AₖAⱼ. Their numeric encoding vectors can be calculated as below:

$$\begin{matrix}{s}_{1} = {A}_{k} {A}_{i} & ( Equation 6 ) \\ {s}_{2} = {A}_{k} {A}_{j} & ( Equation 7 ) \\ {x}_{1} = Ω \times ( Ω \times {β}_{i} + {β}_{k} ) & ( Equation 8 ) \\ {x}_{2} = Ω \times ( Ω \times {β}_{j} + {β}_{k} ) , & ( Equation 9 )\end{matrix}$$

where x₁ and x₂ are the encoding vectors of s₁ and s₂. The Euclidean distance between s₁ and s₂ may be calculated by:

$$\begin{matrix}‖ {x}_{1} - {x}_{2} ‖ = {δ}^{T} δ , where δ = {x}_{1} - {x}_{2} & ( Equation 10 ) \\ = {( {Ω}^{2} ( {β}_{i} - {β}_{j} ) )}^{T} ( {Ω}^{2} ( {β}_{i} - {β}_{j} ) ) & ( Equation 11 ) \\ = {( {β}_{i} - {β}_{j} )}^{T} {Ω}^{T} {Ω}^{T} Ω Ω ( {β}_{i} - {β}_{j} ) . & ( Equation 12 )\end{matrix}$$

The above value may be equal to the distance between amino acid Aᵢ and amino acid Aⱼ, as this is the only mismatch. Thus:

$${( {β}_{i} - {β}_{j} )}^{T} {Ω}^{T} {Ω}^{T} Ω Ω ( {β}_{i} - {β}_{j} ) = {( {β}_{i} - {β}_{j} )}^{T} ( {β}_{i} - {β}_{j} ) \approx {M}_{i j} . \qquad ( Equation 13 )$$

Without losing generality, Ω^{T}Ω=I. In other words, Q may be a unitary matrix. It can be easily shown that longer sequences with one mismatch follow the same pattern above.

In a second example, two amino acids sequences may be off by two consecutive amino acids. The variable s₁ may be defined as s₁=AᵢAⱼ and s₂=AₜAₖ. It can be shown that the distance between the embedding vectors x₁ and x₂ is:

$$\begin{matrix}‖ {x}_{1} - {x}_{2} ‖ = {( {Ω}^{2} ( {β}_{j} - {β}_{k} ) + Ω ( {β}_{i} - {β}_{t} ) )}^{T} ( {Ω}^{2} ( {β}_{j} - {β}_{k} ) + & ( Equation 14 ) \\ Ω ( {β}_{i} - {β}_{t} ) ) & \\ = {( {β}_{j} - {β}_{k} )}^{T} ( {β}_{j} - {β}_{k} ) + {( {β}_{i} - {β}_{t} )}^{T} ( {β}_{i} - {β}_{t} ) - & ( Equation 15 ) \\ 2 {( {β}_{i} - {β}_{t} )}^{T} Ω ( {β}_{j} - {β}_{k} ) & \\ \approx {M}_{j k} + {M}_{i t} - 2 {( {β}_{i} - {β}_{t} )}^{T} Ω ( {β}_{j} - {β}_{k} ) . & ( Equation 16 )\end{matrix}$$

Preferably, the third term to may be zero for ∀i,j,t,k. One solution may be to let Ω be a rotation in [symbol] ^{2r}, by imposing a perpendicular rotation from the first r dimensional space to the complement space. A simple realization may be:

$${Ω}_{2} = ( \begin{matrix}\underline{0} & I \\ I & \underline{0}\end{matrix} ) , \qquad ( Equation 16 )$$

where I may be the r-dimensional identity matrix, and 0 may be an r-dimension zero matrix. In fact, the Ω defined this way may be a representation of order 2 cyclic group G₂. G₂ may have only two elements: e and g, with g²=e. This notation may be useful when in examples with multiple consecutive mismatches. The βᵢ may be extended to [symbol] ^{2r} accordingly, with the first r dimensions filled with the values derived from the MDS embedding, and the remaining dimensions filled with a vector of zeros:

$${β}_{i}^{2 r} = ( \begin{matrix}{β}_{i} \\ 0\end{matrix} ) . \qquad ( Equation 17 )$$

Here 0 may be a vector of zeros with dimension r. The new vector may satisfy:

$${β}_{i}^{2 {r}^{T}} {Ω}_{2} {β}_{j}^{2 r} = ( {β}_{i}^{T} , {0}^{T} ) ( \begin{matrix}\underline{0} & I \\ I & \underline{0}\end{matrix} ) ( \begin{matrix}{β}_{j} \\ 0\end{matrix} ) = ( {β}_{i}^{T} , {0}^{T} ) ( \begin{matrix}0 \\ {β}_{j}\end{matrix} ) = 0. \qquad ( Equation 18 )$$

In a third example, there may be multiple consecutive mismatches. It may be proven that for a sequence sᵢ=Aᵢ_{ₖ}Aᵢ_{ₖ₋₁} . . . Aᵢ_{₁}, k≥3, its encoding vector may be written as:

$${x}_{i} = {\sum }_{l = 1}^{k} {Ω}^{l} {β}_{{i}_{l}} , l = 1 , 2 , … , k . \qquad ( Equation 19 )$$

Consider another sequence, sⱼ=Aⱼ_{ₖ}Aⱼ_{ₖ₋₁} . . . Aⱼ_{₁}:

$${x}_{j} = {\sum }_{l = 1}^{k} {Ω}^{l} {β}_{{j}_{l}} , l = 1 , 2 , … , k . \qquad ( Equation 20 )$$

The distance of xᵢ and xⱼ may be:

$$‖ {x}_{i} - {x}_{j} ‖ = {\sum }_{l = 1}^{k} {( {β}_{{i}_{l}} - {β}_{{j}_{l}} )}^{T} ( {β}_{{i}_{l}} - {β}_{{j}_{l}} ) - 2 {\sum }_{u = 2}^{k} {\sum }_{1 \leq v < u}^{k} {( {β}_{{i}_{u}} - {β}_{{j}_{u}} )}^{T} {( {Ω}^{u} )}^{T} ( {Ω}^{v} ) ( {β}_{{i}_{ν}} - {β}_{{j}_{ν}} ) . \qquad ( Equation 21 )$$

In an ideal scenario, all the terms in the double Σ may be 0, and the distance between sᵢ and sⱼ may be Σₗ₌₁^{k} Mᵢ_{ₗ}ⱼ_{ₗ}. This may require:

$${( {β}_{{i}_{u}} - {β}_{{j}_{u}} )}^{T} {Ω}^{u - v} ( {β}_{{i}_{ν}} - {β}_{{j}_{ν}} ) \qquad ( Equation 22 )$$

being 0 for ∀u,v,k. Or, generally:

$${x}^{T} {Ω}^{u - v} y = 0 for ∀ x , y ∈ {ℝ}^{r} . \qquad ( Equation 23 )$$

There may be no solution for such an Ω in [symbol] ^{r} but similar to the second example, the dimensionality of the embedding space may be increased to kr. In this way, one may be able to construct Ω from n order cyclic group Gₙ, which is an Abel group. However, increased dimensionality may increase the computational complexity in the encoding step by a factor of 0(k). Also, even with the exact solution, the distance calculated by MDS embedding may not perfectly align with BLOSUM62 scores. Therefore, there may be a trade-off to increase dimensionality. In practice, k may be set to 6, which is a reasonable number considering the median length of T cell CDR3 sequences is 14, with the first 4 and last 1 amino acids almost invariant. To construct the corresponding matrix, representation of the element in G₃ may be derived by:

$${Ω}_{3} = ( \begin{matrix}\underline{0} & \underline{0} & I \\ I & \underline{0} & \underline{0} \\ \underline{0} & I & \underline{0}\end{matrix} ) . \qquad ( Equation 24 )$$

Both G₂ and G₃ may be normal subgroups of G₆, with G₆=G₂⊗G₃. Therefore, from Ω₂ and Ω₃, Ω₆ may be constructed:

$${Ω}_{6} = ( \begin{matrix}{0}_{\underline{\_}} & {0}_{\underline{\_}} & {Ω}_{2} \\ {Ω}_{2} & {0}_{\underline{\_}} & {0}_{\underline{\_}} \\ {0}_{\underline{\_}} & {Ω}_{2} & {0}_{\underline{\_}}\end{matrix} ) . \qquad ( Equation 25 )$$

Here 0 is a zero matrix with dimension 2r. Accordingly, the MDS embedding vectors may become:

$${β}_{i}^{6 r} = {( {β}_{i}^{T} , {0}^{T} , {0}^{T} , {0}^{T} , {0}^{T} , {0}^{T} )}^{T} . \qquad ( Equation 26 )$$

With this representation, the terms in the double Σ may be 0 when u−v≤6. When u−v>6 (i.e. the two strings have more than 6 consecutive mismatches), the application of Ω₆ as the transformation matrix may introduce unwanted variance to the final distance. Depending on the vectors on each side of the matrix, the addition may be either positive or negative. However, when comparing CDR3 sequences with more than 6 mismatches, it is usually not important what the exact distance between them is. This is because only the sequences with highest similarities will be selected as antigen-specific TCR clusters, and at the desirable cut-off of the alignment score, the number of mismatches between two CDR3 sequences is usually smaller than 3.

In a fourth example, there may be multiple non-consecutive mismatches. It may be assumed that two sequences of interest, sᵢ and sⱼ, both with length k, differ at the first and last positions, such that sᵢ=Aᵢ_{ₖ}Aᵢ_{ₖ₋₁} . . . Aᵢ_{₂}Aᵢ_{₁}, k≥3 and sⱼ=Aⱼ_{ₖ}Aᵢ_{ₖ₋₁} . . . Aᵢ_{₂}Aⱼ_{₁}, k≥3. The isometric coordinates after transformation are:

$${x}_{i} = {\sum }_{l = 1}^{k} {Ω}^{l} {β}_{{i}_{l}} , l = 1 , 2 , … , k \qquad ( Equation 27 )$$

$${x}_{j} = {\sum }_{l = 2}^{k - 1} {Ω}^{l} {β}_{{j}_{l}} + {Ω}^{k} {β}_{{j}_{k}} + Ω {β}_{{j}_{1}} . \qquad ( Equation 28 )$$

Their distance may be calculated by:

$$‖ {x}_{i} - {x}_{j} ‖ = {( {β}_{{i}_{l}} - {β}_{{j}_{l}} )}^{T} ( {β}_{{i}_{l}} - {β}_{{j}_{l}} ) + {( {β}_{{i}_{k}} - {β}_{{j}_{k}} )}^{T} ( {β}_{{i}_{k}} - {β}_{{j}_{k}} ) - 2 {( {β}_{{i}_{1}} - {β}_{{j}_{1}} )}^{T} {( Ω )}^{T} ( {Ω}^{k} ) ( {β}_{{i}_{k}} - {β}_{{j}_{k}} ) . \qquad ( Equation 29 )$$

This may be similar to the third example (i.e., multiple consecutive mismatches), except that the number of cross terms may be smaller, which may be written as:

$$- 2 {( {β}_{{i}_{1}} - {β}_{{j}_{1}} )}^{T} ( {Ω}^{k - 1} ) ( {β}_{{i}_{k}} - {β}_{{j}_{k}} ) . \qquad ( Equation 30 )$$

When Ω is selected as Ω₆, similar to the third example, the cross term may be 0 as long as NIC is observed. However, ifNIC is violated (i.e., the two mismatches are exactly 6 amino acids apart) the cross term may become non-zero. This term may have an impact on the final outcome. First, if the cross term remains negative (with probability of ½), the estimated isometric distance may be smaller than the exact value. This may not affect the outcome, as the stringent Smith-Waterman alignment may be applied to ensure high sequence similarity. It may be shown that for CDR3s with length 16, with the first 3 and last 2 amino acids clipped, the chance of having two mismatches exactly 6 amino acids apart given there are two mismatches may be

$$\frac{5}{( \begin{matrix}11 \\ 2\end{matrix} )} = 0.091 .$$

This may be the maximum probability among all lengths.

Therefore, violation of NIC may affect, at most, 0.091/2=4.6% of the comparisons with two mismatches. When this happens, somewhat similar sequences may have larger distance and might be excluded from the downstream clustering. To mitigate this effect, a relatively large default isometric distance cutoff (−t 10) may be applied to be inclusive. The current choice of parameterization is a balance between clustering accuracy and computation speed.

The approximate isometric embedding of CDR3 sequences may allow for efficient search of their nearest neighbors (NN) in the Euclidean space for fast clustering. One or more machine learning based classification techniques may be used to perform the NN search.

As understood by those of skill in the art, machine learning based classification techniques may vary depending on the desired implementation, without departing from the disclosed technology. For example, machine learning classification schemes can utilize one or more of the following, alone or in combination: hidden Markov models; recurrent neural networks; convolutional neural networks; Bayesian symbolic methods; general adversarial networks; support vector machines; image registration methods; applicable rule-based system. Where regression algorithms are used, they may include including but are not limited to: a Stochastic Gradient Descent Regressor, and/or a Passive Aggressive Regressor, etc.

Machine learning classification models may also be based on clustering algorithms (e.g., a Mini-batch K-means clustering algorithm), a recommendation algorithm (e.g., a Miniwise Hashing algorithm, or Euclidean LSH algorithm), and/or an anomaly detection algorithm, such as a Local outlier factor. Additionally, machine learning models can employ a dimensionality reduction approach, such as, one or more of a Mini-batch Dictionary Learning algorithm, an Incremental Principal Component Analysis (PCA) algorithm, a Latent Dirichlet Allocation algorithm, and/or a Mini-batch K-means algorithm, etc.

In an example, a python package, such as FAISS, may be used to perform the fast indexed-NN search. To find the nearest neighbor of one of the N numeric vectors in [symbol] ^{r}, the time complexity of FAISS may be 0(rlog(N)).

The coordinates of CDR3s (x) may be divided into neighboring clusters. Before clustering, identical CDR3s may be grouped together. First, for each unique sequence xᵢ, i=1, 2, . . . , N, its nearest neighbor xⱼ, j=1, 2, . . . , N; j≠i may be located. If the distance between xᵢ and xⱼ is within a user-defined cut-off (−t option, thr), the two points may be merged together as a new point, with the centroid

$$\frac{{x}_{i} + {x}_{j}}{2}$$

as the new coordinate. If the distance exceeds the cut-off, both points may be removed from iteration. There may be, at least, two types of removed points: 1) points containing only one CDR3 sequence, and 2) points as a centroid of multiple CDR3s. A CDR3 pre-cluster may be recorded for each of the second type of points. The above steps may be repeated until the number of points reaches zero or does not further decrease. CDR3s with different lengths may be separately clustered. All pre-clusters may be kept for further filtering.

K-mer guided fast Smith-Waterman alignment with TCR variable gene matching may be performed on the CDR3 pre-clusters. CDR3s from a pre-cluster may be highly similar, but they may not qualify as antigen-specific groups because: 1) sequences may not be similar enough due to imperfect isometric embedding, and/or 2) TRBV gene information was not taken into consideration. Accordingly, a filtering step may be performed to select antigen-specific CDR3 clusters based on Smith-Waterman alignment and TRBV gene matching.

The size (m) of pre-clusters may be large and conventional direct pairwise comparison may result in quadratic complexity 0(m²). TRBV information may be applied to reduce cluster size. Specifically, a pre-calculated matrix of alignment scores may be used between a pair of TRBV alleles. For each pair of CDR3 sequences in a pre-cluster, their TRBV alleles may be compared. If the comparison score is above a user-defined (−G option, thr_v), an edge may be added between the two sequences. A depth-first search (DFS) may be performed on the final graph to generate isolated subgraphs, with each subgraph a new pre-cluster. This step may split the original pre-cluster into several smaller ones.

Next, a k-mer approach may be used to perform Smith-Waterman alignment. Each CDR3 sequence may be divided it into consecutive 5-mers. A k-mer dictionary (e.g., in the database 107) may be built to store all the sequences, with keys being unique 5-mers, and the values being the CDR3s that contain the given 5-mer. One mismatch may be allowed in the 5-mer when building the dictionary. For example, sequence CASSGVTEAFF may be indexed under both SSGVT and SSVAT. In this way, CDR3 sequences may be connected into a graph via shared k-mers. For each edge in this graph, Smith-Waterman alignment may be run with BLOSUM62 substitution matrix and the alignment score may be calculated. If the score is below a user-defined cut-off (−S option, thr_s), the edge may be removed. The actual complexity of this step may vary from 0(m) to 0(m²). The worst scenario may be reached when every pair of CDR3s in a pre-cluster share similar k-mer motifs. A DFS may be run on the final graph to generate the final CDR3 clusters and report them as the final output.

In an example, new TCR-seq samples may be queried against the final CDR3 clusters of the existing reference TCR-seq data. After the generation of TCR clusters of an input dataset, GIANA can perform query of additional TCRs to this data (the reference). In the query mode, GIANA may analyze one or more of the query file(s), the reference data, and the clustered reference data.

First, the reference TCR-seq data and query TCR-seq data may be converted into isometric coordinates, as described above. A fast nearest neighbor search (e.g., by FAISS) may then be implemented, but limited to the query TCR-seq data. TCRs with distances smaller than a user-defined cut-off (−t option, thr) may be exported into a separate file (tmp_query.txt). This file may contain all the TCRs that could possibly cluster with the query sequences. GIANA clustering may be performed on this file to generate TCR clusters satisfying the stringent cut-offs for Smith-Waterman alignment. The query TCR clusters may then be merged with the reference clusters in the following way: for each query cluster, if any of the sequence came from an existing cluster in the reference data, the two clusters may be merged. This step is to ensure the inclusion of all the neighboring TCRs in the reference data. There may be two or more conditions when a query cluster may not contain any sequence in the reference cluster: 1) all TCRs in the query cluster were similar, but exclusive to the query sample, and/or 2) the query TCR was similar to some very rare reference TCRs, which were not clustered with any other reference samples in the original clustering. Following either condition, the query cluster may be included in the final output.

In an example, the time cost of the query mode may be evaluated by generating reference data containing 200K, 1M, 2M, 6M and 10M TCRs. Different sizes of the query data may be scanned, including 10K, 20K, 30K, 40K and 50K TCRs. Each query file may be clustered against each of the reference data using, for example, a general purpose computer. Elapsed time may be estimated using the time module of python.

In the GIANAsv process, the easiest way to obtain an isometric representation of a CDR3 string: s=A₁A₂ . . . Aₖ, k≥5 after MDS embedding of the 20 amino acids may be to construct a “stacked vector” (i.e., to concatenate the embedding vectors βᵢ, i=1, 2, . . . , k, in the same order). The stacked vector representation may be x=(β₁^{T}, B₂^{T}, . . . , βₖ^{T})^{T}. This representation may satisfy the desirable qualities of the three examples described above. When focusing only on sequences with six or fewer mismatches, the two approaches may be virtually identical. When CDR3s have more than six mismatches, GIANAsv may be more accurate. However, in GIANAsv the dimension r_{GIANAsv} of the embedding vector may be larger than that of GIANA (r_{GIANA}). For GIANA, r_{GIANA}=6×16=96. For GIANAsv, r_{GIANAsv} may vary with different CDR3 length (typically 12-17 amino acids), which can be 2-3 times larger than r_{GIANA}. Increased dimensionality may result in higher memory burden and longer computational times

The GIANA and GIANAsv processes described above provide a number of improvements over conventional TCR clustering methods (e.g., iSMART, GLIPH2 and TCRdist). For example, the GIANA and GIANAsv processes are not only able to process larger TCR data sets and provide more accurate results, they reduce the amount of computational resources required to generate those results. A comparison using TCR repertoire sequencing data of a healthy donor may bused to demonstrate the improvements of the GIANA and GIANAsv methods described herein. In the comparison, TCR clones may be ordered based on their abundance and the top 10K, 20K, . . . , 100K sequences may be selected. All five methods may be applied to each of the subsamples. GIANA, GIANAsv, iSMART and GLIPH2 may be implemented using default parameters. TCRdist does not provide clustering, and only pairwise distances may be calculated.

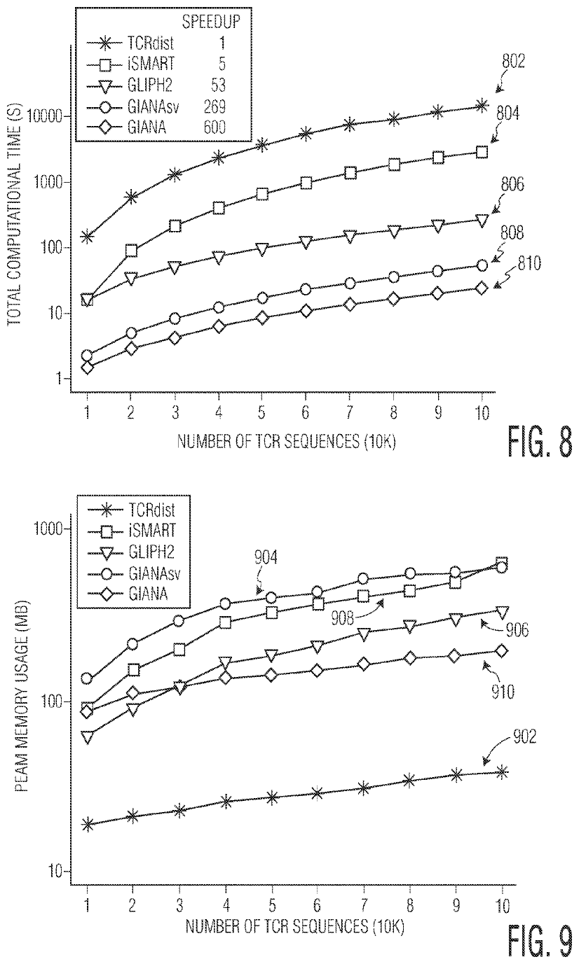

Referring now to FIGS. 8 and 9, charts illustrating the performance of GIANA and GIANAsv against conventional methods are shown. FIG. 8 shows a comparison of time complexity for the different TCR clustering algorithms. The chart shows a total number of TCR sequences analyzed (in 10k increments) on the x-axis and the total computational time (in seconds) on the y-axis. Line 802 shows the performance of TCRdist, line 804 shows the performance of iSMART, line 806 shows the performance of GLIPH2, line 808 shows the performance of GIANAsv, and line 810 shows the performance of GIANA. Speedup may be calculated based on the time cost for the 100K TCR sample.

As shown in FIG. 8, GIANA (line 810) has the lowest time cost throughout the benchmark, taking 23.9 seconds to process 100K sequences, whereas TCRdist (line 802) took 14,338 s. GIANAsv (line 808) is slower than GIANA (line 810) by a factor of 2.2. This is expected because stacked vector encoding resulted in higher dimensionality of the isometric embedding space, and increased the time cost during nearest neighbor search. Notably, GLIPH2 (line 806) is the fastest algorithm behind GIANA (line 810) and GIANAsv (line 808) because it avoids pairwise alignment through motif-guided search. Table 1 below shows a comparison of computational time and memory consumption of GIANA, GIANAsv, iSMART, TCRdist and GLIPH2. In an example, the computations may be performed on a system running macOS Catalina v10.15.2 with a 3.5 GHz Dual-Core Intel Core i7 processor and 16 GB 2133 MHz LPDDR3 memory.

TABLE 1
Comparison of Computational Time and Memory Consumption

| | TCR | 10K | 20K | 30K | 40K | 50K | 60K | 70K | 80K | 90K | 100K |
|---|---|---|---|---|---|---|---|---|---|---|---|
| GIANA | Time (s) | 1.5 | 2.9 | 4.2 | 6.3 | 8.4 | 10.9 | 13.7 | 16.6 | 20.2 | 23.9 |
| | MB | 87 | 109 | 120 | 137 | 142 | 149 | 161 | 175 | 182 | 194 |
| GIANAsv | Time (s) | 2.3 | 5 | 8.3 | 12.2 | 17 | 23 | 29 | 35.4 | 44 | 53.3 |
| | MB | 134 | 212 | 288 | 362 | 394 | 421 | 508 | 540 | 549 | 599 |
| iSMART | Time (s) | 16.5 | 88.8 | 212 | 409 | 657 | 984 | 1380 | 1833 | 2323 | 2850 |
| | MB | 90 | 152 | 197 | 284 | 320 | 362 | 401 | 439 | 484 | 622 |
| TCRdist | Time (s) | 145.9 | 580 | 1300 | 2330 | 3668 | 5411 | 7371 | 9093 | 11695 | 14338 |
| | MB | 19 | 21 | 23 | 26 | 27 | 29 | 31 | 34 | 37 | 38 |
| GLIPH2 | Time (s) | 16.7 | 34.9 | 51.9 | 75.1 | 99.6 | 127.3 | 156.7 | 183 | 224.2 | 271.4 |
| | MB | 63 | 92 | 122 | 165 | 183 | 208 | 247 | 270 | 302 | 334 |

FIG. 9 shows memory usage of the different TCR clustering algorithms when evaluating time complexity. The chart shows a total number of TCR sequences analyzed (in 10k increments) on the x-axis and the peak memory usage (in megabytes) on the y-axis. Line 902 shows the performance of TCRdist, line 904 shows the performance of iSMART, line 906 shows the performance of GLIPH2, line 908 shows the performance of GIANAsv, and line 910 shows the performance of GIANA.

GIANA and GIANAsv may also achieve higher accuracy in predicting antigen-specific TCRs than conventional methods. Antigen-specificity may be the most desirable feature of TCR clustering. An analysis using 61,366 non-redundant known TCR/antigen pairs from the public domain, covering over 900 different epitopes from diverse pathogens was performed. Each method was performed and output clusters from each method were denoted as a “pure cluster” if all the TCRs in an output cluster were specific to only one epitope. Cluster purity may be defined as the percentage of TCRs specific to the most common epitope in a given cluster. A “purecluster” is defined to have purity equals to 1.

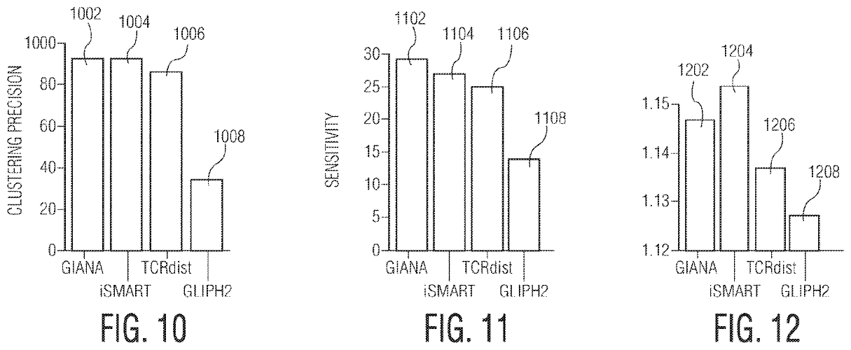

Referring now to FIGS. 10 and 11, charts illustrating comparisons of clustering precision and sensitivity of GIANA as compared to conventional methods are shown. FIG. 10 shows clustering precision on the y-axis, which may be defined as the percentage of pure clusters in the output. GIANA, iSMART, TCRdist, and GLIPH2 are represented by bars 1002, 1004, 1006, and 1008 respectively. As shown by bar 1002, GIANA has the highest precision (93%) across all methods, whereas GLIPH2 has the lowest (35%). FIG. 11 shows clustering sensitivity on the y-axis, which may be defined as the total number of TCRs in all the pure clusters divided by the number of all the testing TCRs. GIANA, iSMART, TCRdist, and GLIPH2 are represented by bars 1102, 1104, 1106, and 1108 respectively. As shown by bar 1102, GIANA also has the highest sensitivity (29%).

Referring now to FIG. 12, a normalized mutual information (NMI) comparison between the four methods is shown. The NMI between TCR clusters and epitope specificity was measured using the same training dataset. Similar levels of NMI across all the methods was observed, though GLIPH2 remained to be the lowest.

Table 2 below shows an evaluation of pure cluster sensitivity and clustering precision for GIANA, iSMART, TCRdist and GLIPH2. A total of 61,366 TCRs with known antigen specificity were used in this analysis. After excluding singleton TCRs (only one sequence per epitope), there were 60,700 remaining.

TABLE 2
Evaluation of Pure Cluster Sensitivity and Clustering Precision

| | GIANA | ISMART | TCRdist | GLIPH2 |
|---|---|---|---|---|
| # Clustered TCRs | 17,250 | 16,828 | 18,383 | 31,563 |
| # Clusters | 7,586 | 7,649 | 7,316 | 11,945 |
| # Pure clusters | 7,289 | 7,387 | 7,130 | 4,333 |
| # Pure TCRs | 16,202 | 16,096 | 15,595 | 11.514 |
| Specificity (Pure | 96.1% | 96.6% | 97.4% | 36.3% |
| clusters/Clusters) | | | | |
| Sensitivity (Pure | 26.7% | 26.5% | 25.6% | 19.0% |
| TCRs/Total | | | | |
| number of TCRs) | | | | |

The fractions for GIANA (96%), iSMART (97%) and TCRdist (97%) are similar, yet substantially lower for GLIPH2 (36%). Pure cluster retention may be defined as the total number of TCRs in all the pure clusters divided by the number of all the testing TCRs. GIANA also has similar level of retention (27%) as other methods, except for GLIPH2 (19%). For the three methods that rely on Smith Waterman alignment (GIANA, iSMART and TCRdist), the impact of a range of alignment score cut-offs (−S option in GIANA) was explored.

Referring now to FIG. 13, a chart illustrating precision-recall curves measuring the performance of GIANA in a range of parameter settings is shown. The y-axis illustrates precision, which is defined as a fraction of true positive calls within a total number of calls. The x-axis illustrates recall, which is a number of true positive calls divided by a number of positive cases in the population. This analysis was applicable to GIANA, TCRdist and iSMART, as all three are based on SW alignment.

At precision>0.95, all three methods share similar curves. The “elbow” shape of the curves is due to the use of pure cluster TCRs in the calculation of recall. When the cutoff is reduced, TCRs from different antigens may get clustered, thus reducing the fraction of pure clusters. A cutoff of 3.6 rather than 3.5 is preferred, as it only slightly reduced the recall (from 0.268 to 0.267), but increased almost 3 percent of precision (from 0.932 to 0.961). Accordingly, the default parameter for −S option in GIANA may be set to 3.6.

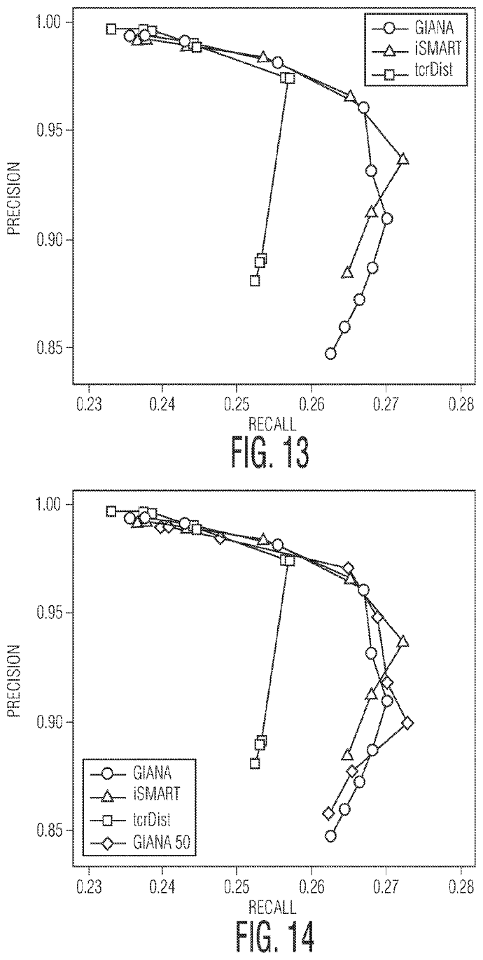

Referring now to FIG. 14, a chart illustrating precision-recall curves comparing the performance GIANA using BLOSUM62 as the substitution matrix for mismatches, iSMART, TCRdist, and GIANA using BLOSUM50 as the substitution matrix for mismatches is shown. As in FIG. 13, the y-axis illustrates precision, which is defined as a fraction of true positive calls within a total number of calls. The x-axis illustrates recall, which is a number of true positive calls divided by a number of positive cases in the population.

The curve labeled “GIANA 50” is the curve for GIANA with BLOSUM50 matrix. This curve is very similar to the curve of the original version of GIANA with BLOSUM62 matrix, labeled “GIANA.” This may be due, in part, to the BLOSUM50 and BLOSUM62 matrices being similar in their off-diagonal values. The differences in the diagonal values were eliminated when transformed into the distance matrix in GIANA. Accordingly, the clustering accuracy of GIANA may be relatively robust to the choice of protein substitution criteria and the choice of using either BLOSUM62 or BLOSUM50 matrices may not materially affect the precision or recall of a final output.

It should be noted that TCRs specific to known epitopes were collected from, among other sources, the Immune Epitope Database and Analysis Resource (IEDB) and the VDJdb online browser. Only TCRβ CDR3 sequences, TRBV genes, and their associated antigens were kept. After removal of redundant or incomplete sequences, a total of 61,366 CDR3s were obtained, covering ˜900 epitopes from diverse pathogens. All methods were applied to the dataset to perform antigen-specific clustering using their default parameters. For TCRdist, the R code was written to perform depth-first search on the sequence pairs with distances smaller than 15. The time complexity calculation for TCRdist does not include the depth-first search to find TCR clusters. This cut-off of 15 has balanced sensitivity and specificity, comparable to that of iSMART4. Choosing a larger cut-off may increase the total number of clustered TCRs, at the cost of lower specificity of each cluster.

As described above, clusters with all the TCRs specific to the same antigen are defined as “pure clusters.” Sensitivity is defined as the total number of TCRs contained in all the pure clusters divided by the total number of sequences (i.e., 61,366). Clustering precision is defined as the number of pure clusters divided by the number of total clusters. These measures were used to compare the performance of antigen-specific clustering of all four methods.

In addition, unlike conventional methods, GIANA is able to retrieve antigen-specific TCRs from real, large and noisy TCR-seq samples using TCRs with known antigen-specificity. From the above benchmark antigen-specific TCRs, those specific to three epitopes expected to be missing in healthy individuals were analyzed: the YAW and YLQ epitopes from the recent outbreak of Severe Acute Respiratory Syndrome Coronavirus-2 (SARS-CoV-2) virus, and the FRD epitope from Human Immunodeficiency Virus-1 (HIV-1). 20% of these TCRs were mixed with 100,000 TCRs from a healthy donor as testing data. The remaining non-overlapping 80% antigen-specific TCRs were used as training data to recover the test sequences. Any sequence clustered with the training data may be identified as a “positive.” True positives are the 20% spiked-in antigen-specific TCRs, whereas false positives are those from the healthy donor.

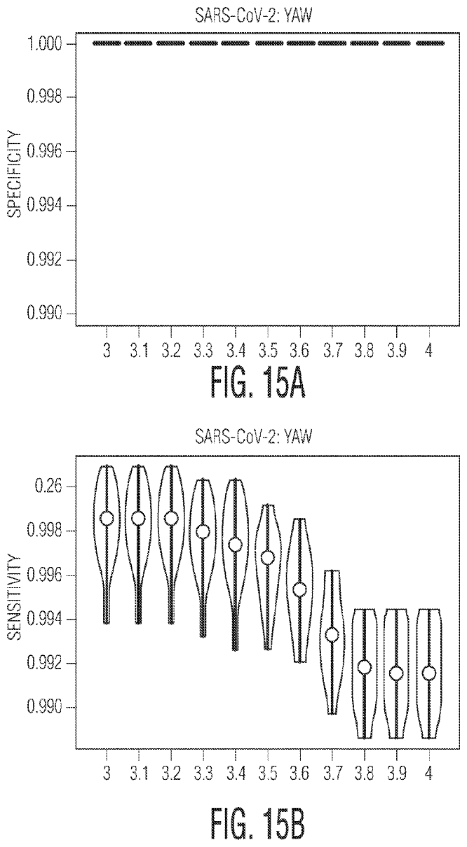

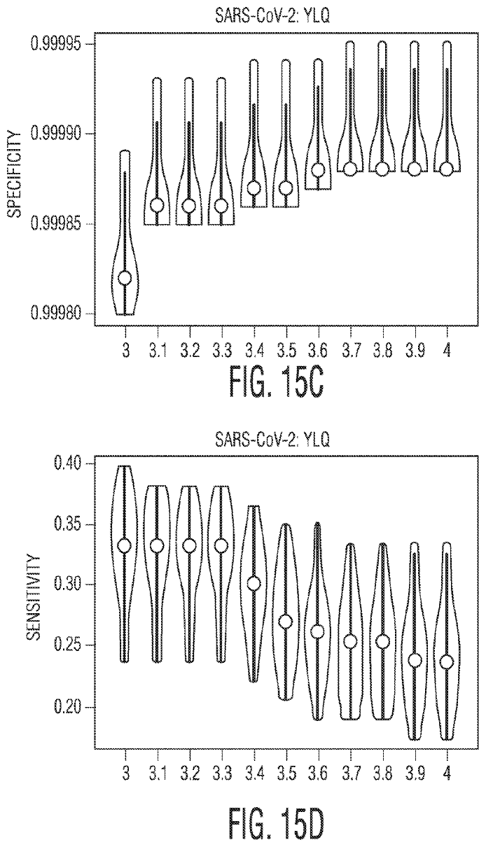

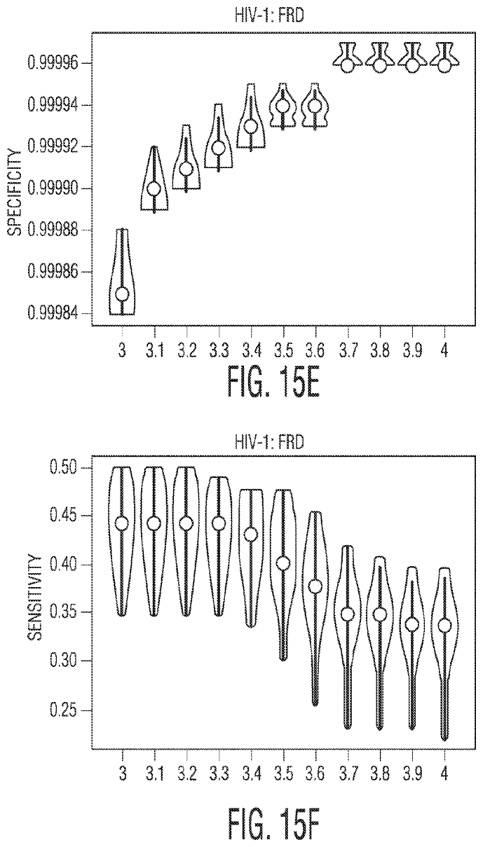

Referring now to FIGS. 15A-15C, charts showing the sensitivity and specificity of GIANA when applied to large and noisy TCR-seq samples is shown. FIGS. 15A and 15B show the specificity and sensitivity of the YAW epitope from SARS-CoV-2. FIGS. 15C and 15D 15B show the specificity and sensitivity of the YLC epitope from SARS-CoV-2. FIGS. 15E and 15F show specificity and sensitivity of the FRD epitope from HIV-1. The violin plots shown in FIGS. 14A-14C illustrate a distribution of the data. The symmetric curve of the side of the “violin” is the actual probability density of the data points. In the middle, a typical box plot illustrates data mean (middle point) and interquartile ranges. The x-axis for each chart is the cut-off for Smith-Waterman Alignment score.

The violin plots shown in FIGS. 14A-14C illustrate a distribution of the data. The symmetric curve of the side of the “violin” is the actual probability density of the data points. In the middle, a typical box plot illustrates data mean (middle point) and interquartile ranges.

The y-axis of each violin plot is either specificity or sensitivity. Specificity is defined as the number of true negatives divided by the total number of algorithm-called negatives. Sensitivity is defined as the number of true positives divided by the total number of algorithm-called positives. Algorithm called positives and negatives are defined using GIANA cluster: a sequence will be called positive if it is clustered with a spiked-in CDR3 of known antigen-specificity.

The x-axis is the cut-off for Smith-Waterman alignment score, a key parameter in GIANA, with a maximum of 4.0. The cut-off is a tunable parameter in GIANA. For example, if the cut-off is set 3.7, any sequence pairs with a Smith-Waterman alignment score (normalized by sequence length, so maximum is 4.0) that is higher than 3.7 would be clustered together. Sequence pairs with lower than 3.7 score will be separated. Higher cut-off results in higher specificity at the cost of reduced sensitivity. For all three epitopes, GIANA achieved over 99.99% specificity at 20%-50% sensitivity.

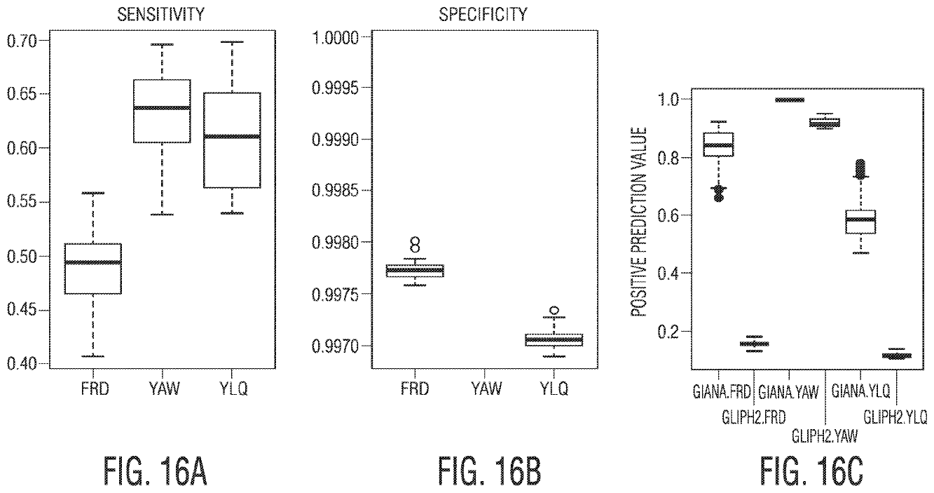

Referring now to FIGS. 16A-16C, charts illustrating the performance of GLIPH2 with large and noisy TCR-seq samples are shown. FIGS. 16A and 16B show sensitivity and specificity estimations on the y-axes for GLIPH2 using the YAW epitope from SARS-CoV-2, the YLC epitope from SARS-CoV-2, and the FRD epitope from HIV-1. FIG. 16C shows positive prediction value (PPV) estimations on the y-axis for YAW epitope from SARS-CoV-2, the YLC epitope from SARS-CoV-2, and the FRD epitope from HIV-1 using GLIPH2 and GIANA. PPV may be defined as the total number of correctly predicted unique TCRs divided by the total number of unique TCRs clustered with the training data. The unique TCRs may be necessary for this analysis because GLIPH2 may place one TCR into multiple clusters. Although GLIPH2 reached higher sensitivity, its specificity is lower than GIANA. More importantly, the PPVs of GIANA reached over 60% for all epitopes, while the PPVs of GLIPH2 for 2 out of the 3 epitopes were lower than 20%.

It should be noted that in silico mixing experiments were performed to assess the performance of GIANA in finding TCRs specific to known antigens. Three antigens were selected that are unlikely exposed to healthy donors: the YAW and YLQ epitopes from the SARS-CoV-2 and the FRD epitope from HIV-1 virus. TCRs specific to each epitope were selected, with redundancy removed. For each antigen, 20% of TCRs (testing data) were randomly sampled and mixed with the 100K sequences from the healthy donor. There was no overlap between the remaining 80% of antigen-specific TCRs (training data) and the testing data. The mixed sample was considered to be a pseudo-patient carrying the corresponding pathogen. The mixed sample was combined with the training data, and GIANA was applied with Smith-Waterman alignment score cut-off (thr_s) ranging from 3.0 to 4.0 (0.1 increment). For each epitope and parameter setting, in silico mixing was performed 20 times to capture the variations in the data.

From the resulting data, the prediction performance was evaluated. The TCR clusters with at least one TCR were selected from the training data. All the TCRs in these clusters, excluding training data, were positive calls. All TCRs that were not co-clustered with any training TCR were negative calls. True positive calls were defined as sequences labeled as “testing data,” whereas true negative calls were sequences from the original 100K TCRs of the healthy donor. Specificity was defined as the number of true negative calls divided by 100K. Sensitivity was defined as the number of true positive calls divided by the total number of testing TCRs.

In addition, unlike conventional methods, the high speed and specificity of GIANA allows for a query module to cluster new TCR samples with an existing reference dataset, a function that is missing in all current tools.

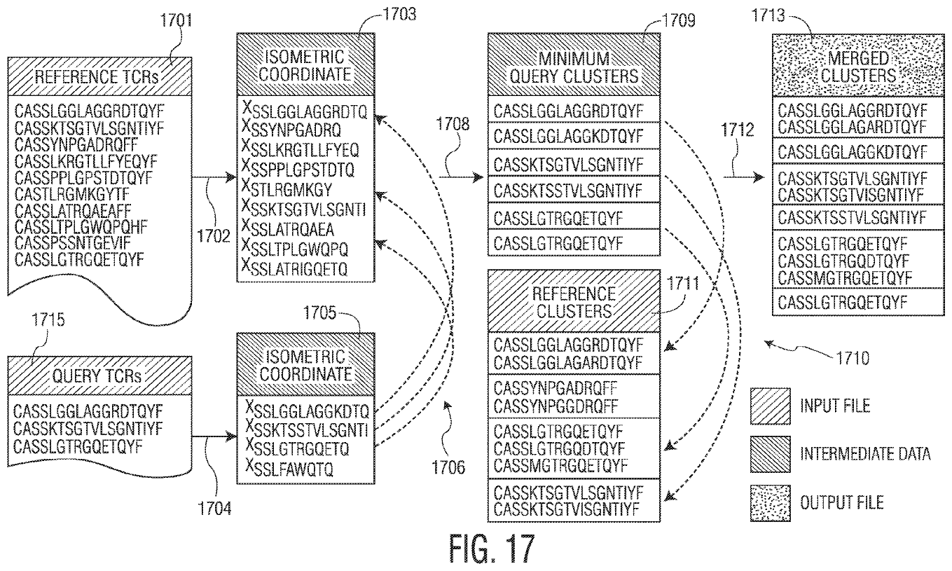

Referring now to FIG. 17, a diagram illustrating fast GIANA query based on isometric transformation is shown. As described above, reference and query TCRs may be transformed into Euclidean space with linear complexity, searched for the nearest neighbors of each query sequence, processed into TCR clusters, and merged with the reference data. The dashed arrows shown in FIG. 17 implicate search directions.

In step 1702, reference TCR-seq data 1701 may be encoded into numeric vectors through a sequence of unitary transformations and each encoded CDR3 sequence may be projected to high-dimensional Euclidean space to form reference isometric coordinates 1703. In step 1704, query TCR-seq data 1715 may be encoded into numeric vectors through a sequence of unitary transformations and each encoded CDR3 sequence may be projected to high-dimensional Euclidean space to form query isometric coordinates 1705. In step 1706, a nearest neighbor search may be performed between the reference isometric coordinates 1703 and the query isometric coordinates 1705. In step 1708, minimum query clusters 1709 may be formed. In step 1710, a nearest neighbor search may be performed between the minimum query clusters 1709 and reference clusters 1711. In step 1712, merged clusters 1713 may be formed.

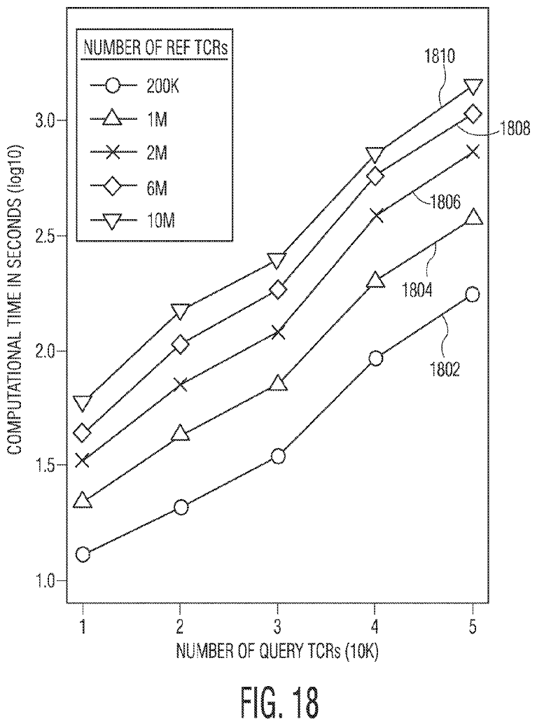

Referring now to FIG. 18, a chart illustrating a time complexity evaluation of GIANA query module using reference/query data with different number of TCRs is shown. The x-axis shows a number of query TCRs in 10k increments, and the y-axis shows a logarithmic representation of computational time in seconds. Lines 1802, 1804, 1806, 1808, and 1810 show 200k reference TCRs, 1M reference TCRs, 2M reference TCRs, 6M reference TCRs, and 10M references TCRs respectively. As shown, GIANA is extremely efficient: it took approximately 176 seconds to query 104 TCRs against 107 reference sequences, a task with computational load equal to 100 billion pairwise comparisons. Table 3 below shows computational time consumption of GIANA query of TCR samples with different sizes. Time was measured in seconds.

TABLE 3
Computational Time Consumption of GIANA
query of TCR Samples with Different Sizes

| | | Query TCR Number | | | | |
|---|---|---|---|---|---|---|
| | | 10K | 20K | 30K | 40K | 50K |
| Reference | 200K | 13 | 22 | 33 | 44 | 60 |
| TCR | 1M | 21 | 43 | 72 | 107 | 151 |
| Number | 2M | 35 | 71 | 121 | 184 | 249 |
| | 6M | 93 | 200 | 387 | 578 | 719 |
| | 10M | 176 | 379 | 732 | 1,066 | 1,438 |

This type of repertoire classification is an important task with immediate applications to disease diagnosis and prognosis. Typically, this task has been approached by multiple instance learning or deep learning. The common limitation of these methods is their high computational cost that prevents from scaling up to large TCR-seq datasets.

GIANA query may be used to classify TCR repertoires. For example, three reference datasets with 20, 100, or 200 TCR-seq samples may be evenly split into COVID-19 patients and healthy controls (HC). An additional 154 COVID-19 and 120 HC samples may be queried to each of the references.

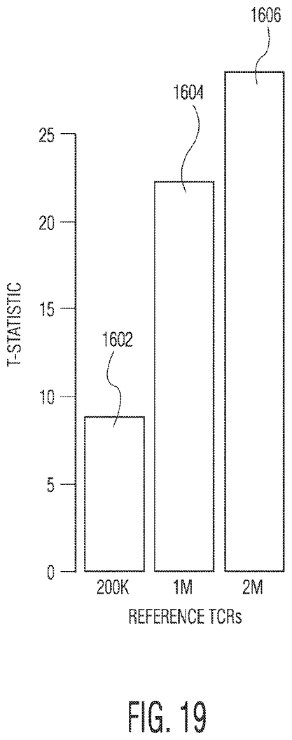

Referring now to FIG. 19, a chart illustrating a degree of separation of query COVID-19 patients from healthy controls by clustering against the reference datasets is shown. The number of TCRs of the reference data is shown as x-axis labels. The t-statistic on the y-axis was produced using the t.test function, to perform two sample t-test using the COVID-19 fractions to separate the COVID-19 and HC query samples. Bars 1902, 1904, and 1906 show 200k reference TCRs, 1M reference TCRs, and 2M reference TCRs respectively. Two sample t-tests were performed using the COVID-19 fractions estimated from the query data to obtain the t-statistics. All p values were significant at the level of 2.2×10⁻¹⁶. For each query sample, a fraction of TCRs co-clustered with COVID-19 reference patients may be calculated. This fraction may be significantly higher for the COVID-19 patients in the query samples, with increasing separation from query HCs as the size of reference data increases. Using this fraction as a predictor, an increasing area under the receiver operation Curve (AUC) is observed for reference with larger sample size.

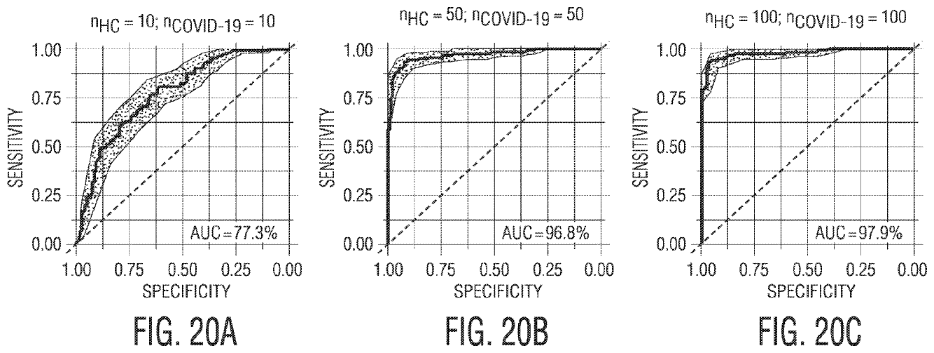

Referring now to FIGS. 20A-20C, charts illustrating receiver operating characteristic (ROC) curves using COVID-19 fraction as the single predictor. The numbers of COVID-19 and HC samples are shown above each chart, with each sample containing 10K TCR sequences.

The ROC curve is an unbiased way to visualize the prediction power of a given method. Here, the COVID-19 fraction is used as a continuous predictor. By changing the threshold of this fraction, the specificity (x-axis) and the sensitivity (y-axis) will change. FIG. 20A shows 10 HC samples and 10 COVID-19 samples. FIG. 20B shows 50 HC samples and 50 COVID-19 samples. FIG. 20C shows 100 HC samples and 100 COVID-19 samples. 95% confidence intervals were estimated from 2,000 stratified bootstraps. Notably, with 2 million reference TCRs, the sensitivity (79%) and specificity (100%) of this approach surpassed some existing tests for COVID-19, suggesting the potential utilities of this approach in disease diagnosis. More importantly, the accuracy of repertoire classification improved with more reference samples. This is likely due to the sharing of disease-specific TCRs across different patients, which are usually shared at low frequencies, and thus a larger reference data will result in higher clustering probability, smaller dispersion and better precision.

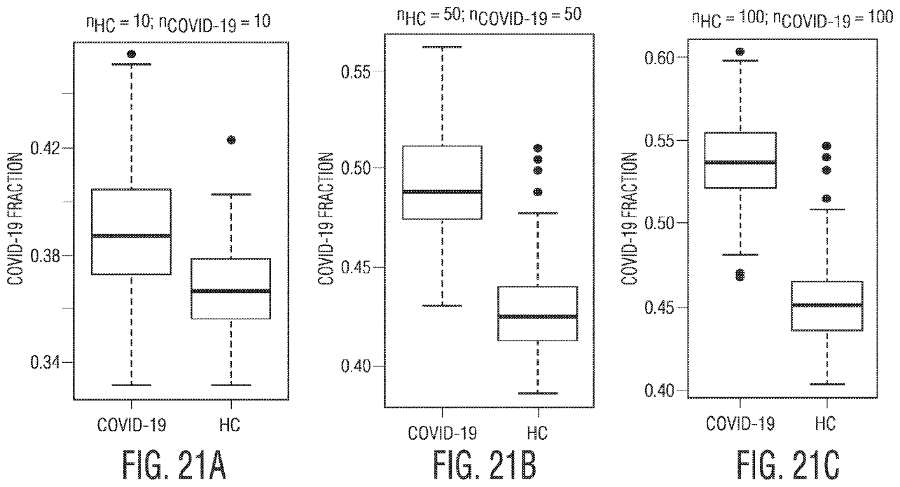

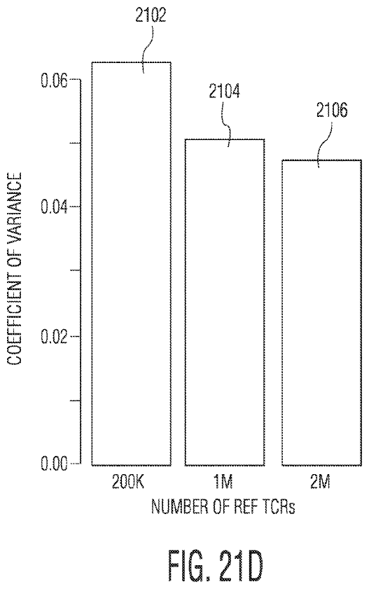

Referring now to FIGS. 21A-21D, charts illustrating the original COVID-19 fraction scores for both COVID-19 patients and healthy donors and a coefficient of variance of COVID-19 fractions with different number of reference TCRs are shown. FIGS. 21A-21 show the distribution of TCR fractions co-clustered with COVID-19 reference samples under different reference data configurations. FIG. 21A shows the 10 HC samples and 10 COVID-19 samples. FIG. 21B shows the 50 HC samples and 50 COVID-19 samples. FIG. 21C shows the 100 HC samples and 100 COVID-19 samples.

In FIG. 211D, the x-axis shows the number of reference TCRs and the y-axis shows the coefficient of variance. The coefficient of variance may be defined as the standard deviation divided by the mean of COVID-19 fractions of the COVID-19 patients in the query samples. Bars 2102, 2104, and 2106 show 200k reference TCRs, 1M reference TCRs, and 2M reference TCRs respectively. A decreasing coefficient of variance of COVID-19 fractions is seen with more reference samples.

The above features can be demonstrated with an even larger reference dataset. For example a dataset containing 10 million TCRs, which consisted of 1,213 samples from cancer, COVID-19, multiple-sclerosis (MS) patients and HCs was used. GIANA was applied to perform antigen-specific clustering of all 10M TCRs and investigate the similarity of different repertoire samples measured by the level of shared TCR clusters. Table 4 shows TCR-seq sample cohorts used as the reference data.

TABLE 4
TCR-Seq Sample Cohorts Used as Reference Data

| Disease | | | Sample | Unique | |
|---|---|---|---|---|---|
| Type | Cohort | Disease | Size | Samples | PMID |
| Healthy | Emerson et | Healthy | 100 | 100 | 28369038 |
| Control | al., 2017 | Control | | | |
| | | (batch1) | | | |
| Multiple | Emerson et | Multiple | 50 | 25 | 23428915 |
| Sclerosis | al., 2013 | Sclerosis | | | |
| COVID-19 | Nolan et | COVID-19 | 311 | 311 | 32793896 |
| | al., 2020 | (Adaptive, | | | |
| | | ISB) | | | |
| Cancer | Snyder et | Bladder | 117 | 30 | 28552987 |
| | al., 2017 | Cancer | | | |
| | Mansfield et | Lung Cancer | 40 | 20 | 29391594 |
| | al., 2018 | and Brain | | | |
| | | Metastasis | | | |
| | Sims et | Glioma | 32 | 15 | 27261081 |
| | al., 2016 | | | | |
| | Page et | Breast | 63 | 16 | 31831558 |
| | al., 2019 | Cancer | | | |
| | Reuben et | Lung Cancer | 121 | 121 | 32001676 |
| | al., 2019 | | | | |
| | Emerson et | Ovarian | 96 | 5 | 24027095 |
| | al., 2013 | Cancer | | | |
| | Stromnes et | Pancreatic | 16 | 16 | 29066497 |
| | al., 2017 | Cancer | | | |
| | Tumeh et | Melanoma | 34 | 23 | 25428505 |
| | al., 2014 | | | | |
| | Le et al., | Colorectal | 35 | 3 | 28596308 |
| | 2017 | Cancer | | | |
| | Duhen et | Head and | 33 | 8 | 30006565 |
| | al., 2018 | Neck, | | | |
| | | Ovarian and | | | |
| | | Melanoma | | | |
| | Chow et | Renal Cell | 53 | 26 | 32900949 |
| | al., 2020 | Carcinoma | | | |
| | Riaz et | Melanoma | 58 | 29 | 29033130 |
| | al., 2017 | | | | |
| | Sherwood et | Colorectal | 14 | 14 | 23771160 |
| | al., 2013 | Cancer | | | |

For some cohorts, not all the available samples were used when creating the reference data. For each sample, top 10,000 most abundant TCRs were selected, and if the data contained fewer than 10,000 sequences, all were used. Unique samples indicated the number of independent patients involved in the study. Sample size recorded the number of total TCR-seq samples in that cohort that were used in the reference. Emerson 2017 cohort contained 666 healthy donors in batch 1, from which 100 samples were randomly selected. The COVID-19 cohort contained over 1,400 patients, assembled from multiple international COVID-19 studies. Two cohorts were collected by Adaptive Biotechnology (Adaptive, n=154) and Institute for System Biology (ISB, n-157), respectively. In an example, GIANA took 19.5 hours to cluster the reference data on a high performance computing cluster with 8 CPUs and 128G memory.

Referring now to FIGS. 22A-22D, graphics representation of similarities of the TCR-seq samples based on TCR co-clustering is shown. In FIGS. 22A-22D, physical proximity represents similarity, so TCR-seq samples (shown as dots) that are closer together are more similar. To generate FIGS. 22A-22D, a sample-wise count-sharing matrix was computed from original TCR clustering results of the 1,213 reference samples described above. A spearman correlation matrix was also calculated based on counts of co-clustered TCRs, with pairs having a correlation value ≤0.4 set to zero. The resulting sparse matrix was used to generate the graph. Nodes with fewer than 2 connections were removed to visualize the sample groups.

FIG. 22A is an overall view of a first cluster of cancer patients (shown in detail FIG. 22B), a second cluster of HCs and MS patients (shown in detail in FIG. 22C), and a third cluster of lung cancer patients and COVID-19 patients (shown in detail in FIG. 22D). FIG. 22A shows clear separations of most cancer patients in the first cluster from HCs and MS patients in the second cluster. Interestingly, lung cancer patients and COVID-19 patients formed the separate third cluster. It is known that local inflammatory conditions, such as viral infection or cancer, may release tissue-resident T cells into the circulation, a likely cause for TCR repertoire sharing. These findings further suggest that in the lung tissue, the magnitude of T cell exodus might be high enough to transcend disease types.

It should be noted that to test the feasibility of repertoire classification using TCR clustering, 10, 50 and 100 COVID-19 samples were combined with 10, 50 and 100 healthy controls to generate 3 reference datasets with 20, 100, and 200 samples. Each sample contained 10K TCRs, selected by ranking the clonal abundance. Query samples contained 154 COVID-19 patients and 120 HCs. There was no overlap between query and reference samples. The TCR clusters were generated for each query sample using GIANA. For each sample, TCR clusters with more than 100 samples were removed, as these TCRs were likely generated from small-world connections and not informative to disease specificity. For the remaining clusters, the fraction of reference TCRs contributed by the COVID-19 patients was calculated and used as the predictor.

In the multiple disease classification task, 712 cancer, 311 COVID-19, 25 MS patients, and 100 HC samples were combined to produce a reference data of 10M TCRs. Another 62 cancer, 193 COVID-19, 12 MS, and 153 HC samples were collected to make the query, assuming the disease labels were unknown. Similar analysis was performed for each query cluster file to estimate the fractions of each disease category, including HC. These fractions were used to predict diseases and perform the ROC analysis. Specifically, HC fractions were used for all the comparisons with HC samples. As an exploratory approach, for pairwise separation of the 3 diseases, the difference between the two disease fractions was used. For example, when predicting cancer from MS patients, Cancer Fraction-MS Fraction was used as the predictor.

In addition, unlike conventional methods, GIANA may be used as a novel multi-disease detection platform through ultra-large-scale TCR clustering and querying. The ultra-large-scale clustering by GIANA allows for the inspection of disease-specific vs. tissue-specific TCRs. In an example, TCR clusters in the lung cancer and COVID-19 patients were divided into 3 categories: i) COVID-19 specific; ii) lung cancer specific; iii) shared between the two diseases.

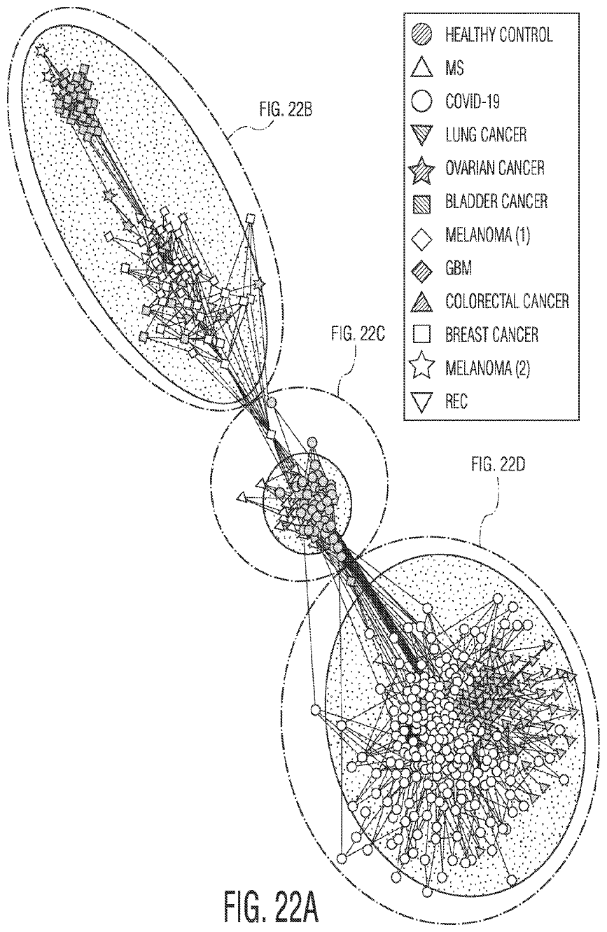

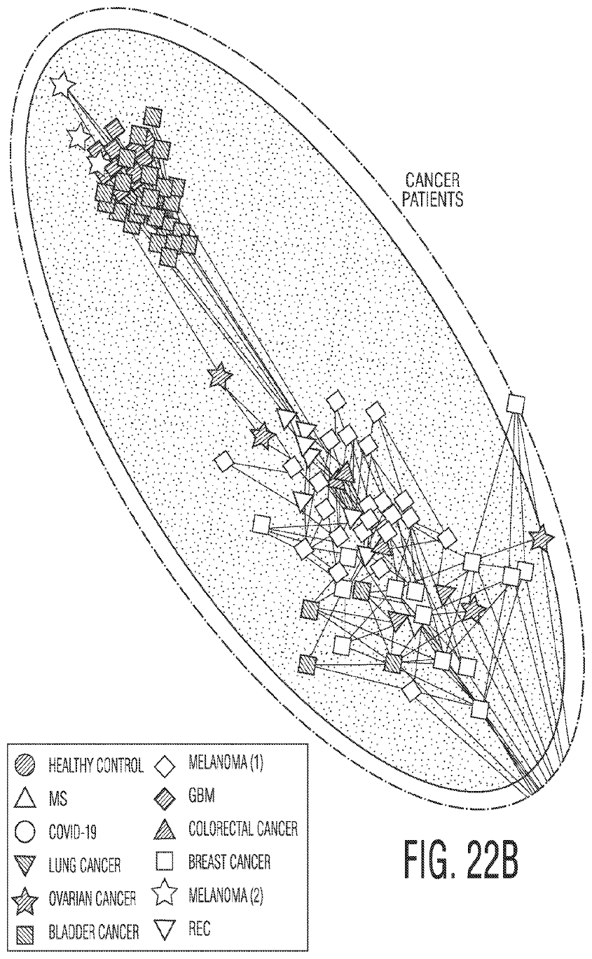

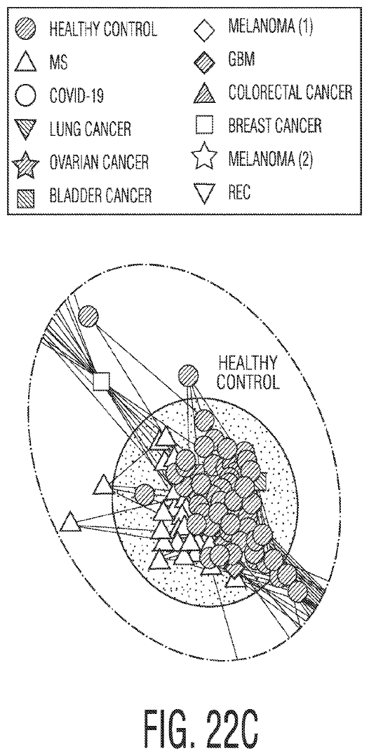

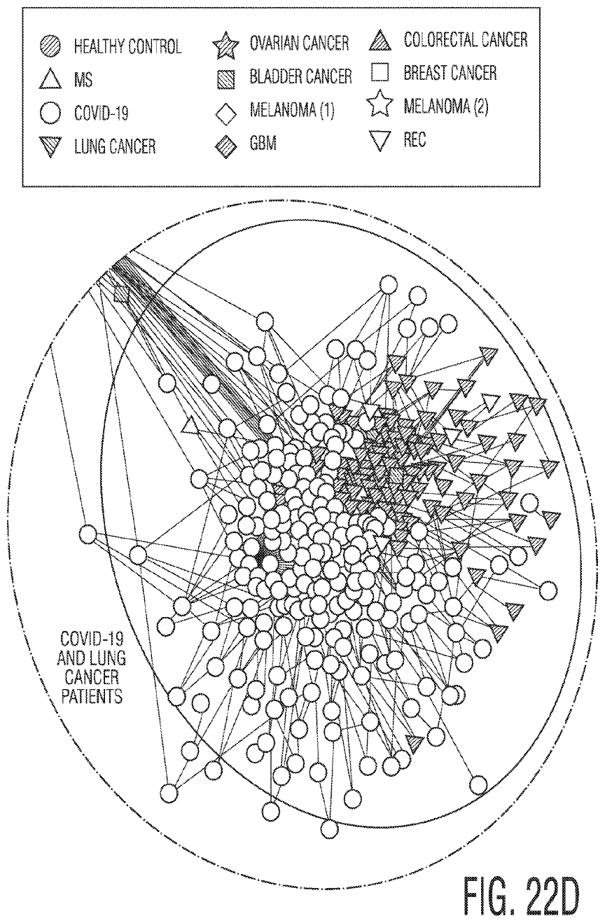

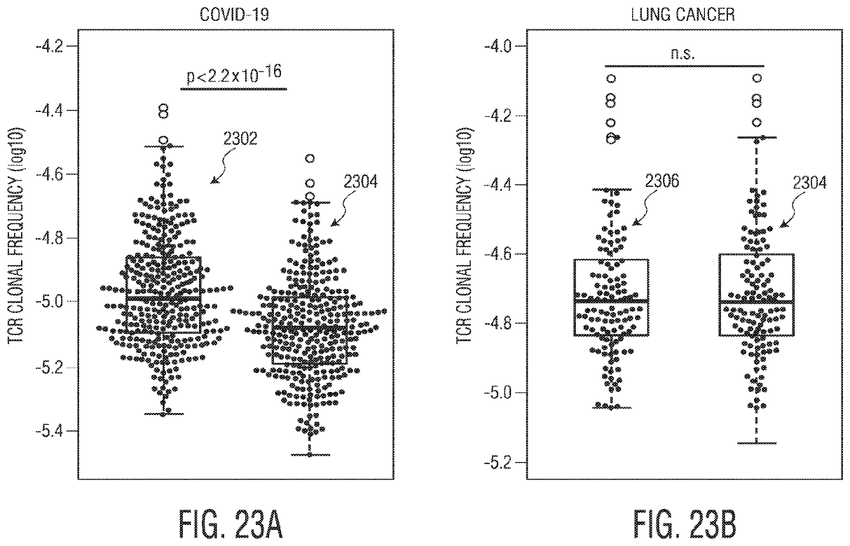

Referring now to FIGS. 23A-23B, beeswarm plots illustrating the distributions of TCR clonal frequencies of different categories are shown. The beeswarm plots are box plots showing actual data points, which are the TCR clonal frequency (y-axis). TCR clonal frequency is defined as the percentage of sequencing reads of a given TCR divided by the total number of reads in a sample. The x-axis for both charts is a side-by-side comparison of two sample categories.

FIG. 23A show a comparison between TCRs that were only found in patients with COVID-19 2302 and TCRs that were found in both COVID-19 and lung cancer patients 2304. FIG. 23B shows a comparison between TCRs that were that were only found in patients with lung cancer 2306 and the TCRs that were found in both COVID-19 and lung cancer patients 2304. For the TCRs that were found in both COVID-19 and lung cancer patients 2304, clonal frequencies were chosen to match the cohort of the disease-specific TCRs. The p value in FIG. 23A (p<2.2e-16) is a measure the type-I error of a statistical test. The p value in FIG. 23B (n.s.) means not significant, which is a p value larger than 0.05.

As shown in FIGS. 23A-23B, there is a significantly higher clonal frequency of category i) vs iii) for COVID-19 patients, whereas there is no difference between category ii) and iii) for the lung cancer patients. TCR frequencies were matched within same cohort to avoid batch effect, and thus the higher abundance of COVID-19 specific TCRs is likely caused by an immune response to SARS-CoV-2. Indeed, only COVID-19 specific TCRs underwent dynamic regulation after viral infection, which peaked within the first 2 weeks post-exposure, and decreased afterwards.

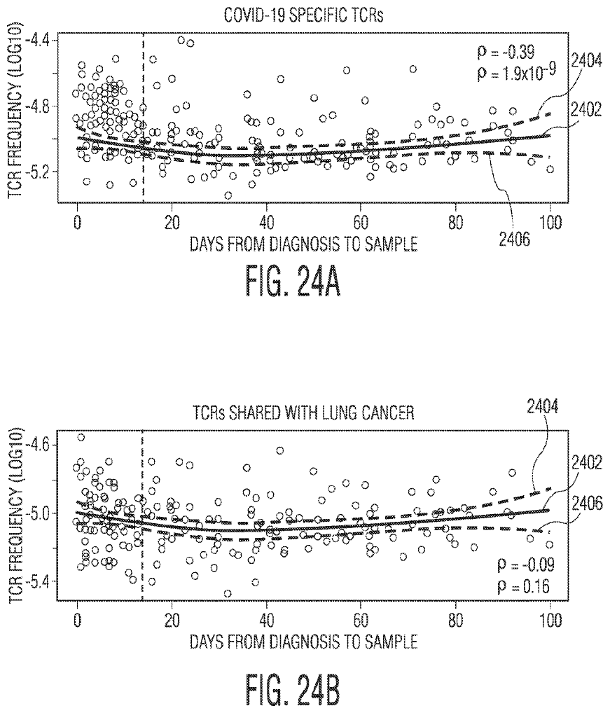

Referring now to FIGS. 24A-24B, graphs illustrating dynamic changes of TCR clonal frequencies during the course of SARS-CoV-2 infection are shown. In both charts, the x-axis shows days from diagnosis to sample collection and the y-axis shows a logarithmic representation of TCR frequency. The middle solid line 2402 is the smoothed average of the data. At each time point (x-axis), there are multiple observations of TCR clonal frequency, and the solid line is their averaged value. Likewise, the upper dashed line 2404 is the upper bound of the 95% confidence interval of the observed data. The lower dashed line 2406 is the lower bound of the confidence interval. The “p values” are the type-I error of a Spearman's correlation test performed for this analysis. The Spearman's correlation value (rho) is displayed as the first line within each figure.

Clonal abundance of shared TCRs were unaffected by the timeline after SARS-CoV-2 infection. These figures show that clustering on large TCR repertoire samples may reveal a plethora of shared disease-specific TCRs, which may provide a finer solution to repertoire classification.

Clustered TCRs may be used as markers to assign repertoire samples into multiple diseases by, for example, implementing a leave-one-out validation approach. Specifically, for a given sample, fractions of TCRs co-clustered with cancer, COVID-19, MS patients, or healthy controls in the reference cohort may be calculated, excluding the sample itself. This method may yield 4 class fractions for each sample, which added up to 1. Using the HC fraction, patients may be separated from healthy donors.

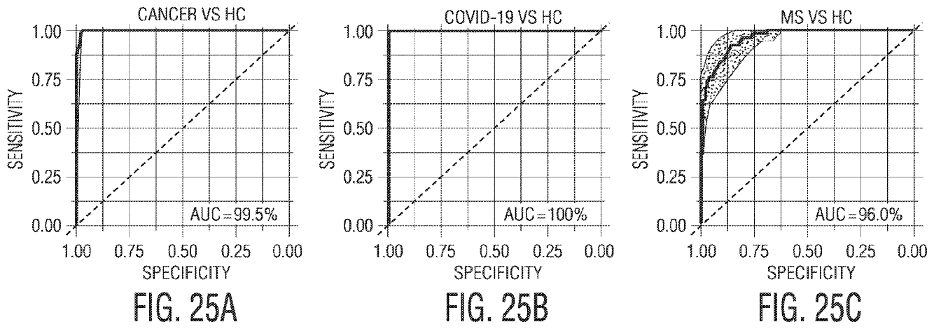

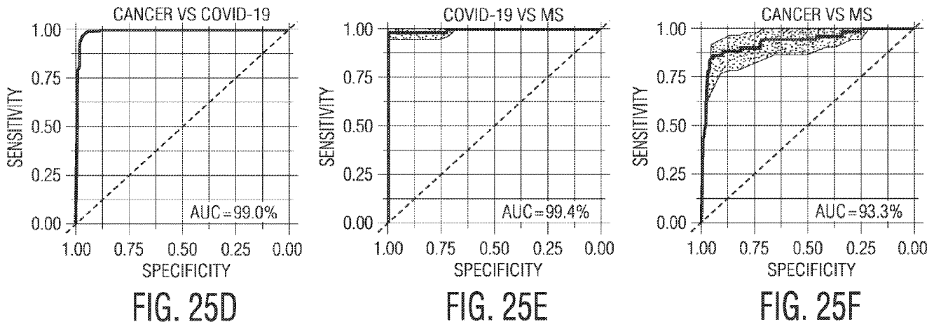

Referring now to FIGS. 25A-25F, charts illustrating ROC curves using the leave-one-out validation approach for disease fractions calculated from co-clustered TCRs are shown. The AUC values are shown at the bottom right of each chart. Each chart shows specificity on the x-axis and sensitivity on the y-axis. FIG. 25A shows a comparison between cancer TCR clusters and HC TCR clusters. FIG. 25B shows a comparison between COVID-19 TCR clusters and HC clusters. FIG. 25C shows a comparison between MS clusters and HC clusters. FIG. 25D shows a comparison between cancer TCR clusters and COVID-19 clusters. FIG. 25E shows a comparison between COVID-19 clusters and MS clusters. FIG. 25F shows a comparison between cancer clusters and MS clusters. 95% confidence intervals may be calculated using 2,000 stratified bootstraps. Near perfect accuracies were observed for all 3 diseases. To differentiate a pair of diseases, the differences between the two corresponding fractions may be used as the predictor, which leads to high (≥93%) AUC values.

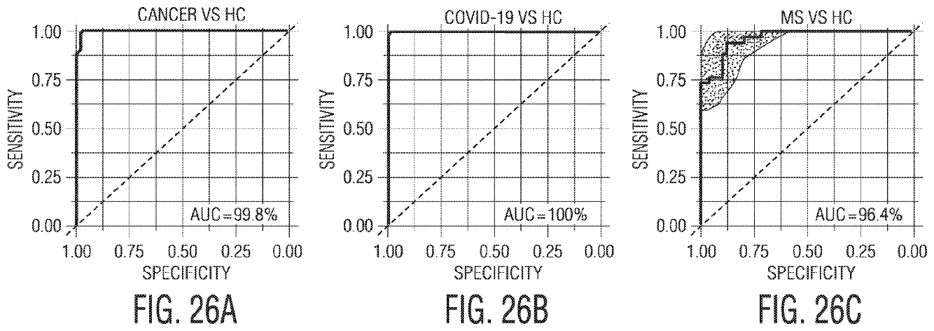

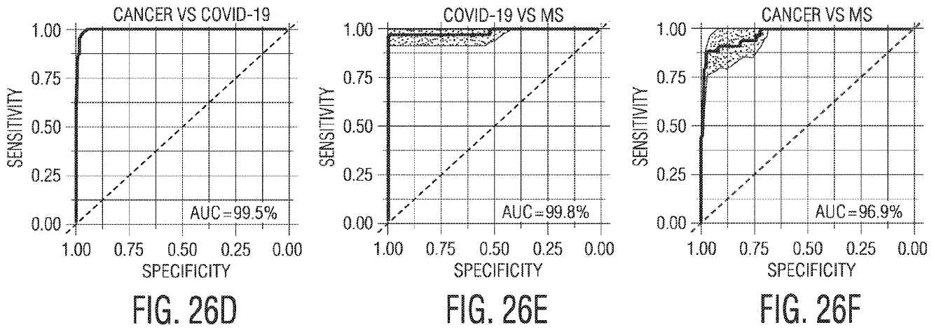

Referring now to FIGS. 26A-2AF, charts illustrating ROC curves using a more stringent approach for disease fractions calculated from co-clustered TCRs are shown. In an example, 40% of the reference samples were randomly selected as the training data, leaving the remaining 60% as test data. Training samples are labeled with “COVID-19,” “Cancer,” “MS,” or “HC.” Each test data was co-clustered with all the training samples to calculate the fraction of TCRs clustered with each sample category. The fraction of “HC” is used to distinguish diseased vs healthy individuals. Other fractions are used to differentiate the three diseases. Similar levels of prediction accuracy as the leave-one-out-validation were achieved by this more stringent method.

The AUC values are shown at the bottom right of each chart. Each chart shows specificity on the x-axis and sensitivity on the y-axis. FIG. 25A shows a comparison between cancer TCR clusters and HC TCR clusters. FIG. 25B shows a comparison between COVID-19 TCR clusters and HC clusters. FIG. 25C shows a comparison between MS clusters and HC clusters. FIG. 25D shows a comparison between cancer TCR clusters and COVID-19 clusters. FIG. 25E shows a comparison between COVID-19 clusters and MS clusters. FIG. 25F shows a comparison between cancer clusters and MS clusters.

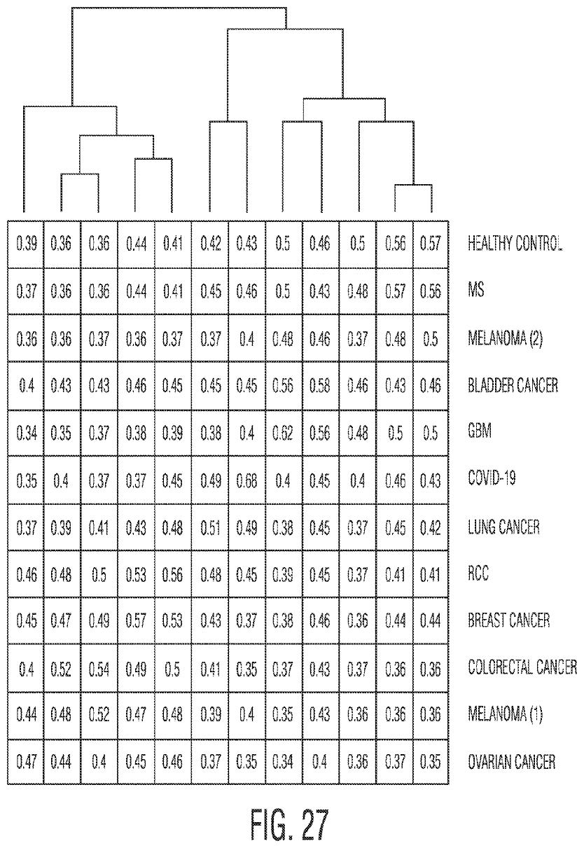

Referring now to FIG. 27, a chart illustrating cross-cohort similarity of reference TCR-seq samples is shown. Using TCR clustering data with N samples, the percentage of TCRs of each sample co-clustered with each of the other samples may be calculated. The self-co-clustering percentage may be assigned to be zero to make all the vectors length N. A Spearman correlation matrix may be calculated from the N-by-N co-clustering fraction matrix. The matrix may then be collapsed according to cancer type. The mean of the top 5 highest correlations are displayed as a heatmap in FIG. 27. Same disease correlations (diagonal values) may be calculated the same manner, except that self-correlations of each sample may be excluded prior to the calculations. Color coding signifies the values on display. For example, red colors represent positive correlations.

The ability to distinguish lung cancer from COVID-19 was not contradictory to the apparent grouping of the two diseases, because within-disease similarity was still higher. However, as most of the diseases were derived from only one study, it raised the concern that the predictability might be contributed by unknown batch effects.

To test out this possibility, GIANA was used to predict the disease labels of unseen samples from independent cohorts. GIANA was applied to query 267 new TCR-seq samples of the same diseases and 153 HC samples against the reference dataset. All samples were derived from peripheral blood. The same approach was used to calculate the fractions of TCRs co-clustered with reference cancer, COVID-19, MS, or HC sequences. Table 5 below shows TCR-seq sample cohorts used as the query data.

TABLE 5
TCR-Seq Sample Cohorts Used as the Query Data

| Disease | | | Sample | Unique | |
|---|---|---|---|---|---|
| Type | Cohort | Disease | Size | Samples | PMID |
| Healthy | Emerson et | Healthy | 120 | 120 | 28369038 |
| Control | al., 2017 | Control | | | |
| | | (batch2) | | | |
| | DeWitt et | Active | 33 | 33 | 29914888 |
| | al., 2018 | Tuberculosis | | | |
| Multiple | Bertoli et | Multiple | 12 | 6 | 31719595 |
| Sclerosis | al., 2019 | Sclerosis | | | |
| COVID-19 | Nolan et | COVID-19 | 193 | 193 | 32793896 |
| | al., 2020 | (HUniv120) | | | |
| Cancer | Beshnova et | Ovarian, | 25 | 25 | 32817363 |
| | al., 2020 | Pancreatic | | | |
| | | and Renal | | | |
| | | Cancer | | | |
| | Robert et | Melanoma | 21 | 21 | 24583799 |
| | al., 2014 | | | | |
| | Beausang et | Breast | 16 | 16 | 29138313 |
| | al., 2017 | Cancer | | | |

All 120 of the second batch of healthy donors from the Emerson 2017 study were used as control. To avoid overlap with the reference, for the COVID-19 patients, the Hospital Universitario 12 de Octubre (HUniv120, n=193) cohort from the Nolan 2020 study was used. The patients in this cohort were collected from Madrid, Spain. In an example, it took GIANA 20.5 hours to finish the query of all 420 samples on a MacBook Pro with a 3.5 GHz Dual-Core Intel Core i7 processor and 16 GB 2133 MHz LPDDR3 memory.

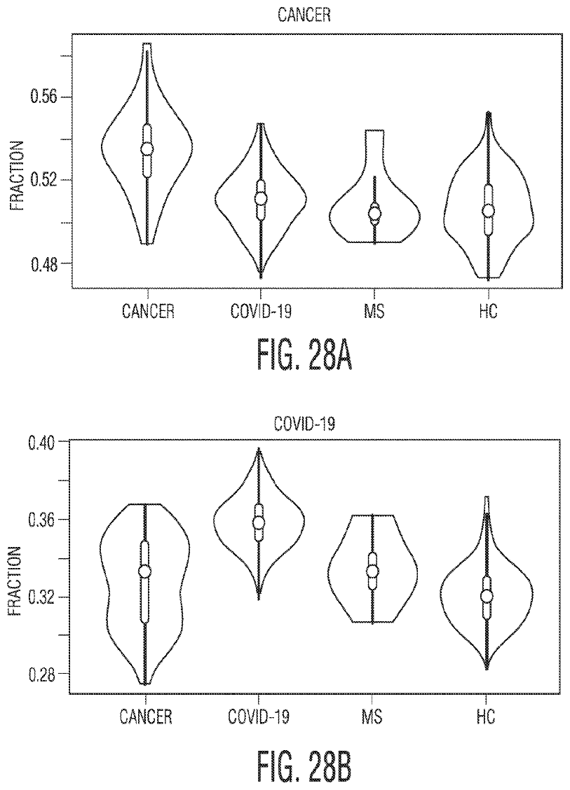

Referring now to FIGS. 28A-28D, “violin plots” showing the distribution of class fractions of cancer, COVID-19, MS patients, and HCs are shown. Each of the “violin plots” shown in FIGS. 28A-28D illustrate a distribution of the data. The symmetric curve of the side of the “violin” is the actual probability density of the data points. In the middle, a typical box plot illustrates data mean (middle point) and interquartile ranges. The y-axis shows the fractions of the TCRs of the given disease category (shown as the title of the subpanel). The x-axis shows the disease categories. FIGS. 28A-28D show that the GIANA estimated TCR fraction of a given disease is the highest in the patients bearing that disease, which justifies its use of a multi-disease predictor.

The class fraction (e.g., the cancer fraction) may be calculated as the proportion of query TCRs clustered with reference TCRs from the cancer patients. Without any model training, this simple approach may distinguish each sample category from the others. HC fractions may distinguish all 3 diseases at over 91% accuracy.

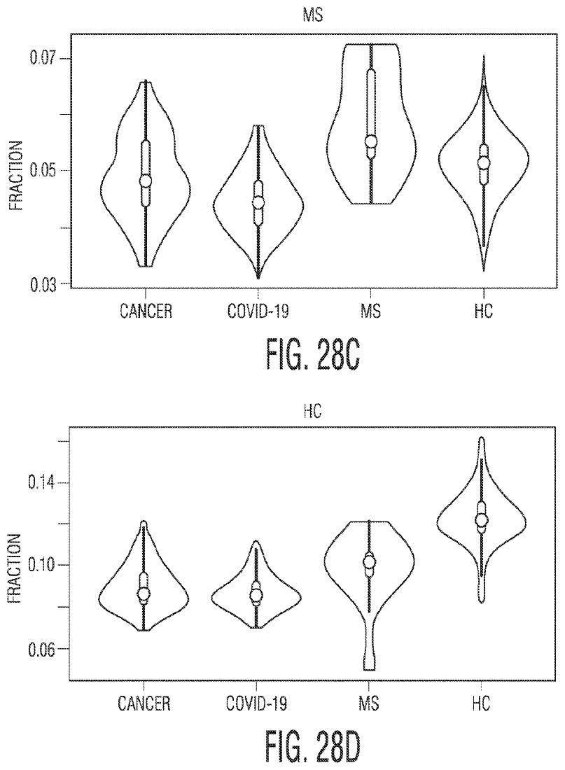

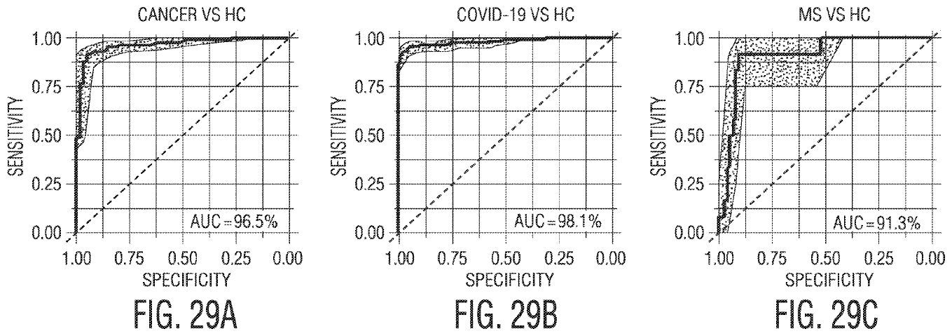

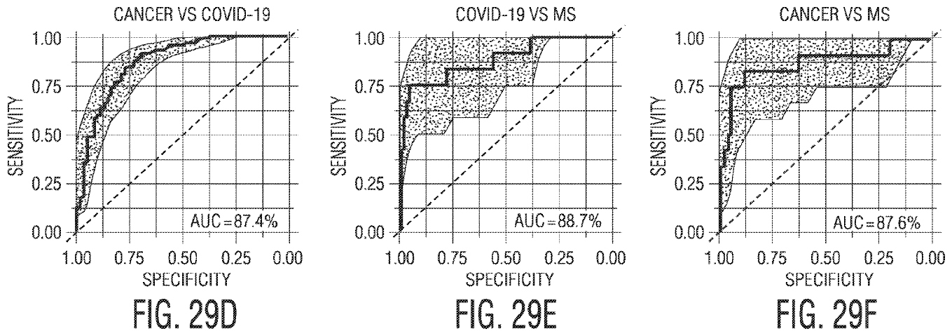

Referring now to FIGS. 29A-29F, charts illustrating ROC curves using disease class fractions as single predictor for pairwise separation of the 4 disease classes are shown. The fraction may be the percentage of TCRs co-clustered with a given class of samples in the reference dataset. The AUC values are shown at the bottom right of each charts. Each chart shows specificity on the x-axis and sensitivity on the y-axis. FIG. 29A shows a comparison between cancer TCR clusters and HC TCR clusters. FIG. 29B shows a comparison between COVID-19 TCR clusters and HC clusters. FIG. 29C shows a comparison between MS clusters and HC clusters. FIG. 29D shows a comparison between cancer TCR clusters and COVID-19 clusters. FIG. 29E shows a comparison between COVID-19 clusters and MS clusters. FIG. 29F shows a comparison between cancer clusters and MS clusters. 95% confidence intervals were calculated using 2,000 stratified bootstraps. Pairwise separation between diseases all reached above 87% AUCs. Since the query samples were derived from studies not included in the reference data, the high AUCs were not caused by unknown batch or cohort-specific effects, thus likely reflected the real predictability for the three diseases.

The GIANA query performance was compared to conventional repertoire classification methods based on multiple instance learning (MIL) and the fitting of cohort-specific parameters (e.g., DeepRC and others). While GIANA does not require any parameter fitting, the conventional methods provide suitable reference data that has similar attributes as the query samples (e.g., repertoire samples from true COVID-19 patients and negative controls). Using a cohort containing both HCMV+ and HCMV− subjects, 75% of the samples were applied as a reference (similar as training) with the remaining 25% applied as test data. Each test sample was queried against the reference data. For each query sample, the fraction of TCRs co-clustered with HCMV+reference subjects was calculated and used as a predictor. This simple approach reached 83.06% AUC, the same DeepRC and better than other methods, as shown in the chart below. Therefore, GIANA may be a competitive method for repertoire classification.

TABLE 6
AUC for GIANA vs. Conventional Methods

| Method | AUC |
|---|---|
| GIANA | 0.831 |
| DeepRC | 0.831 |
| SVM (MM) | 0.825 |
| SVM (J) | 0.546 |
| KNN (MM) | 0.679 |
| KNN (J) | 0.534 |
| MIL (KMER) | 0.582 |
| MIL (TCRB) | 0.515 |

It should be noted that the pROC package of the R programming language was used to generate the ROC curves and estimate the AUC values above, with 95% confidence intervals computed by 2,000 stratified bootstrap replicates, implemented using the ci.auc function in the pROC package. FIG. 22 was generated using the igraph package. The heatmap with annotated values of FIG. 27 was produced using heatmap.2 function in the gplots package. For all the boxplots displayed in the figures, the middle line defines the median value, with borders of the boxes indicating the 25% (Q1) and 75% (Q3) quartiles of the data. Lower and upper whiskers corresponded to Q1-1.5IQR and Q3+1.5IQR, where IQR is short for inter-quartile range.

In summary, GIANA is a novel antigen-specific TCR clustering algorithm that is able to efficiently handle tens of millions of sequences. It achieved higher sensitivity and precision than all existing methods, and is able to retrieve TCRs specific to known antigens with high accuracy. The ultra-large-scale TCR clustering and fast query of novel samples also enabled a novel reference-based repertoire classification framework. GIANA can also analyze single cell RNA-seq data with TCR regions solved, and it is possible to query TCRs from the scRNA-seq data against the large database of TCR repertoire samples in the public domain, and provide new insights over shared antigen-specificity. With minimum modifications, GIANA is applicable to cluster or query large B cell receptor sequencing data as well. Furthermore, the mathematical framework to perform isometric embedding may provide an alternative solution to the classic short DNA or protein sequence alignment problems in the future.

It should be noted that HLA alleles were not considered in GIANA, as such data is unavailable in most current studies. With HLA typing included, the accuracy of TCR clustering and query methods is expected to improve. Although GIANA does not support gap alignment, it has better sensitivity than the other methods with this functionality. This is because allowing gaps may reduce clustering specificity and compromise the prediction accuracy.

Also, as described above, the simple fraction estimation is used to assign disease classes. With more data, this effort may be improved by machine learning models to optimize the prediction accuracy. Additionally, all cancer patients were compared with other diseases together without differentiating cancer localizations. However, it is contemplated that the power to separate cancer types using enough relevant TCR-seq samples as the reference. Although the current GIANA method already achieves high accuracy of repertoire classification, the diagnostic values of this platform may improve with prospectively collected patient samples.

As demonstrated in autoimmune and infectious diseases, antigen-specific public TCRs shared at low frequencies are potentially important biomarkers which may be detected by comparing large amount of TCRs from thousands of individuals. Methods have been developed to individually detect cancer, COVID-19, and MS using immune repertoire, but none has been able to simultaneously diagnose and separate different diseases. In contrast, GIANA may be used as a unified platform to diagnose infectious disease, autoimmune disorders, and cancer.

This provides a number of improvements over conventional methods. Disease diagnosis has traditionally been mainly symptom-driven, with each disease requiring a distinct set of signatures obtained from diverse clinical assays, such as radioactive imaging, liquid biopsy, invasive endoscopy, surgery, etc. The feasibility of using the immune system as a single biomarker to indicate multiple diseases may shift the paradigm from symptom-driven to immune-response-driven, which may provide a universal solution to many immune-related disorders.

Additionally, differential diagnosis is usually a clinical challenge, and it is anticipated that adding more diseases to the platform will reduce the diagnostic specificity. However, the predication accuracy of GIANA actually increases with the inclusion of more TCR-seq samples.

Further, as immune responses are usually ahead of any measurable symptoms, the GIANA platform has the potential to detect diseases at its early stages, where most diseases are curable or easy to manage. This has already been shown for cancer diagnosis, and the principle of immune regulation also applies to autoimmune disorders, such as MS. Finally, since this platform only requires a small amount of blood to perform targeted V(D)J capture, it may serve as a non-invasive test at low cost. Together, GIANA may be widely used to find antigen-specific TCR clusters, to retrieve sequences specific to known pathogens, such as SARS-CoV-2, and to facilitate disease diagnosis with the fast growing body of TCR data in cancer, immunology and clinical studies.

For the purposes of this disclosure a module is a software, hardware, or firmware (or combinations thereof) system, process or functionality, or component thereof, that performs or facilitates the processes, features, and/or functions described herein (with or without human interaction or augmentation). A module may include sub-modules. Software components of a module may be stored on a computer readable medium for execution by a processor. Modules may be integral to one or more servers, or be loaded and executed by one or more servers. One or more modules may be grouped into an engine or an application.

Those skilled in the art will recognize that the methods and systems of the present disclosure may be implemented in many manners and as such are not to be limited by the foregoing examples. In other words, functional elements being performed by single or multiple components, in various combinations of hardware and software or firmware, and individual functions, may be distributed among software applications at either the client level or server level or both. In this regard, any number of the features of the different examples described herein may be combined into single or multiple examples, and alternate examples having fewer than, or more than, all of the features described herein are possible.

Functionality may also be, in whole or in part, distributed among multiple components, in manners now known or to become known. Thus, a myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the examples of methods presented and described as flowcharts in this disclosure are provided by way of example in order to provide a more complete understanding of the technology. The disclosed methods are not limited to the operations and logical flow presented herein. Alternative examples are contemplated in which the order of the various operations is altered and in which sub-operations described as being part of a larger operation are performed independently.

While various examples have been described for purposes of this disclosure, such examples should not be deemed to limit the teaching of this disclosure to those examples. Various changes and modifications may be made to the elements and operations described above to obtain a result that remains within the scope of the systems and processes described in this disclosure.

What is claimed is:

1. A method of improving computational efficiency for T-cell receptor (TCR) comparisons, the method comprising:
identifying, by a computing device, complementary determining region 3 (CDR3) sequences from a reference TCR sequence (TCR-seq) dataset, the reference TCR-seq dataset consisting of TCRs specific to only one epitope;
encoding, by the computing device, each of the CDR3 sequences from the reference TCR-seq dataset into numeric vectors, the numeric vectors corresponding to a sequence of amino acids in each of the CDR3 sequences;
converting, by the computing device, the numeric vectors to coordinates in a high-dimensional Euclidean space;
generating, by the computing device, a predictive model using a neural network by:
learning, by the neural network, to generate a tree data structure of the numeric vectors based on relative distances of the coordinates, and
grouping, by the neural network, the coordinates into pre-clusters based on the relative distances;
filtering, by the computing device, the CDR3 sequences in the pre-clusters; and
identifying, by the computing device, antigen-specific CDR3 clusters from the filtered pre-clusters.

2. The method of claim 1, further comprising:
performing, by the computing device, the identifying, encoding, converting, generating, and filtering steps on a query TCR-seq dataset, the query TCR-seq dataset having no known antigen-specific TCR information;
comparing, by the computing device, the filtered pre-clusters from the query TCR-seq dataset to the antigen-specific CDR3 clusters; and
determining, by the computing device, that the filtered pre-clusters from the query TCR-seq dataset match the antigen-specific CDR3 clusters to diagnose and/or determine disease status.

3. The method of claim 1, further comprising:
grouping, by the computing device, CDR3 sequences having identical coordinates together.

4. The method of claim 1, wherein the filtering comprises:
comparing, by the computing device, TCR variable (TRBV) alleles of each pair of CDR3 sequences in the pre-clusters to determine an alignment score; and
splitting, by the computing device, the pre-clusters into one or more new pre-clusters if the score is above a predetermined level.

5. The method of claim 4, wherein the filtering further comprises:
performing, by the computing device, a Smith-Waterman alignment on each of the pre-clusters to determine an alignment score; and
removing, by the computing device, a pre-cluster if the score is below a predetermined level.

6. The method of claim 1, wherein the encoding comprises:
performing, by the computing device, a sequence of unitary transformations on each of the CDR3 sequences.

7. A computing device comprising:
a processor operatively coupled to a memory storing non-transitory computer-readable instructions that, when executed by the processor, cause the processor to:
identify complementary determining region 3 (CDR3) sequences from a reference TCR sequence (TCR-seq) dataset, the reference TCR-seq dataset consisting of TCRs specific to only one epitope;
encode each of the CDR3 sequences from the reference TCR-seq dataset into numeric vectors, the numeric vectors corresponding to a sequence of amino acids in each of the CDR3 sequences;
convert the numeric vectors to coordinates in a high-dimensional Euclidean space;
generate a predictive model using a neural network by:
learning, by the neural network, to generate a tree data structure of the numeric vectors based on relative distances of the coordinates, and
grouping, by the neural network, the coordinates into pre-clusters based on the relative distances;
filter the CDR3 sequences in the pre-clusters; and
identify antigen-specific CDR3 clusters from the filtered pre-clusters.

8. The computing device of claim 7, wherein the computer-readable instructions, when executed by the processor, further cause the processor to:
perform the identifying, encoding, converting, generating, and filtering steps on a query TCR-seq dataset, the query TCR-seq dataset having no known antigen-specific TCR information;
compare the filtered pre-clusters from the query TCR-seq dataset to the antigen-specific CDR3 clusters; and
determine that the filtered pre-clusters from the query TCR-seq dataset match the antigen-specific CDR3 clusters to diagnose and/or determine disease status.

9. The computing device of claim 7, wherein the computer-readable instructions, when executed by the processor, further cause the processor to:
group CDR3 sequences having identical coordinates together.

10. The computing device of claim 7, wherein the filtering comprises:
comparing, by the computing device, TCR variable (TRBV) alleles of each pair of CDR3 sequences in the pre-clusters to determine an alignment score; and
splitting, by the computing device, the pre-clusters into one or more new pre-clusters if the score is above a predetermined level.

11. The computing device of claim 10, wherein the filtering further comprises:
performing, by the computing device, a Smith-Waterman alignment on each of the pre-clusters to determine an alignment score; and
removing, by the computing device, a pre-cluster if the score is below a predetermined level.

12. The computing device of claim 7, wherein the encoding comprises:
performing, by the computing device, a sequence of unitary transformations on each of the CDR3 sequences.

13. A non-transitory computer-readable storage medium tangibly encoded with computer-executable instructions, that when executed by a processor associated with a computing device, cause the processor to:
identify complementary determining region 3 (CDR3) sequences from a reference TCR sequence (TCR-seq) dataset, the reference TCR-seq dataset consisting of TCRs specific to only one epitope;
encode each of the CDR3 sequences from the reference TCR-seq dataset into numeric vectors, the numeric vectors corresponding to a sequence of amino acids in each of the CDR3 sequences;
convert the numeric vectors to coordinates in a high-dimensional Euclidean space;
generating a predictive model using a neural network by:
learning, by the neural network, to generate a tree data structure of the numeric vectors based on relative distances of the coordinates, and
group, by the neural network, the coordinates into pre-clusters based on the relative distances;
filter the CDR3 sequences in the pre-clusters; and
identify antigen-specific CDR3 clusters from the filtered pre-clusters.

14. The non-transitory computer-readable storage medium of claim 13, wherein the computer-executable instructions, when executed by the processor, cause the processor to:
perform the identifying, encoding, converting, generating, and filtering steps on a query TCR-seq dataset, the query TCR-seq dataset having no known antigen-specific TCR information;
compare the filtered pre-clusters from the query TCR-seq dataset to the antigen-specific CDR3 clusters; and
determine that the filtered pre-clusters from the query TCR-seq dataset match the antigen-specific CDR3 clusters to diagnose and/or determine disease status.

15. The non-transitory computer-readable storage medium of claim 13, wherein the computer-executable instructions, when executed by the processor, cause the processor to:
group CDR3 sequences having identical coordinates together.

16. The non-transitory computer-readable storage medium of claim 13, wherein the filtering comprises:
comparing, by the computing device, TCR variable (TRBV) alleles of each pair of CDR3 sequences in the pre-clusters to determine an alignment score; and
splitting, by the computing device, the pre-clusters into one or more new pre-clusters if the score is above a predetermined level.

17. The non-transitory computer-readable storage medium of claim 16, wherein the filtering further comprises:
performing, by the computing device, a Smith-Waterman alignment on each of the pre-clusters to determine an alignment score; and
removing, by the computing device, a pre-cluster if the score is below a predetermined level.

18. The non-transitory computer-readable storage medium of claim 13, wherein the encoding comprises:
performing, by the computing device, a sequence of unitary transformations on each of the CDR3 sequences.

19. A method of organizing and querying a T-cell receptor (TCR) database using common antigen specificity, the method comprising:
performing a nearest neighbor search using one or more TCR dissimilarity metrics to find pairs of TCRs with common antigen specificity.

20. The method of claim 19, wherein the one or more TCR dissimilarity metrics comprise one or more of a Smith-Waterman distance and an embedding in a high-dimensional Euclidean space; or any other distance or dissimilarity metric.
